(19) United States
(12) Patent Application Publication
(10) Pub. No.: US 2026/0022411 A1
(43) Pub. Date: Jan. 22, 2026

(54) MICROBIAL CELL FACTORIES PRODUCING THIAMINE

(71) Applicant: BIOSYNTIA APS, Copenhagen (DK)

(72) Inventors: Carlos Guillermo ACEVEDO-ROCHA, Copenhagen (DK); Daniel BONIFACIO, Copenhagen (DK); Benjamin CADDELL, Copenhagen (DK); Hans GENEE Jasper, Copenhagen (DK); David LENNOX-HVENEKILDE, Copenhagen (DK); Bo SALOMONSEN, Copenhagen (DK)

(21) Appl. No.: 18/993,760

(22) PCT Filed: Jul. 12, 2023
(86) PCT No.: PCT/EP2023/069269

§ 371 Date: Jan. 13, 2025

(30) Foreign Application Priority Data
Jul. 15, 2022 (EP) 22185261.9

(51) Int. Cl. C12P 17/16; A61K 31/51; A61K 35/741; A61K 35/742; C07K 14/195; C12N 9/06; C12N 9/10; C12N 9/12; C12N 9/88

(52) U.S. Cl. CPC C12P 17/167; A61K 31/51; A61K 35/741; A61K 35/742; C07K 14/195; C12N 9/0022; C12N 9/1085; C12N 9/1205; C12N 9/1229; C12N 9/1241; C12N 9/13; C12N 9/88; C12Y 104/03019; C12Y 205/01003; C12Y 207/01049; C12Y 207/04016; C12Y 207/07073; C12Y 208/0101; C12Y 401/99017; C12Y 401/99019

(57) ABSTRACT

The present disclosure relates to a genetically modified host cell having improved production of thiamine, wherein the host cell expresses one or more heterologous ThiO enzymes converting glycine into dehydroglycine (DHG) in the host cell and/or one or more heterologous ThiI enzymes catalyzing the transfer of sulfur from IscS to the sulfur carrier protein ThiS in the host cell, whereby the production of the thiamine in the genetically modified host cell is improved compared to an unmodified parent host cell.

### TECHNICAL FIELD

The present disclosure relates to genetically modified host cells having increased production of thiamine through providing for increased production of dehydroglycine (DHG) and ThiS thiocarboxylated protein, to genetic constructs for expression of such mutants; to cultures of the genetically modified host cells and its use to produce the said products; to fermentation liquids comprising the said products resulting from such production; to compositions comprising the fermentation liquid; to dietary, flavour or pharmaceutical preparations made from such compositions and to the uses of such compositions and preparations.

### BACKGROUND

In nature, thiamine is produced by some microbes and plants. The biosynthetic pathway for thiamine in natural organisms is generally well described in the art and the pathway is shown in FIG. 1.

Today, thiamine is industrially produced by using a chemical synthesis process based on non-renewable fossil fuels, and there is no fermentation-based production available. Yet there is a need for developing bio-based processes from renewable sources like sugar that are more sustainable.

Since microorganisms just need tiny amounts of thiamine, the big challenge is to engineer microbes to produce large amounts of this vitamin.

The use of microorganism-based cell factories is a potential route for the biosynthetic production of B vitamins (Acevedo-Rocha, et al. 2019). The advantages of recombinant microorganisms such as E. coli as a microbial cell factory for the production of bio-products are widely recognized due to the fact that: (i) E. coli has unparalleled fast growth kinetics; with a doubling time of about 20 minutes when cultivated in glucose-salts media and under optimal environmental conditions, (ii) it easily achieves a high cell density; where the theoretical density limit of an E. coli liquid culture is estimated to be about 200 g dry cell weight/L or roughly 1×10¹³ viable bacteria/mL. Additionally, there are many molecular tools and protocols at hand for genetic optimization of E. coli; as well as it being an organism that is amenable to the expression of heterologous proteins; both of which may be essential for obtaining high-level production of desired bio-products.

Thiamine is produced in nature by a multistep enzymatic pathway that can be divided into 2 branches (FIG. 1): Pyrimidine (PYR) and thiazole (THZ). In the PYR branch, the radical SAM phosphomethylpyrimidine synthase ThiC converts the substrate 5-aminoimidazole ribonucleotide (AIR) to the pathway intermediate 4-amino-5-hydroxymethyl-2-methylpyrimidine-phosphate (HMP-P), which is phosphorylated to HMP-PP by the kinase ThiD.

In the THZ branch, there are 3 sub-branches that form the thiazole ring. The first, second and third sub-branches, respectively, encompass a sugar backbone coming from pyruvate and glyceraldehyde 3-phosphate catalyzed by Dxs, from a sulfur transfer step coming from cysteine catalyzed by IscS, ThiI, ThiF, ThiS, and from dehydroglycine (DHG) that can be generated from tyrosine by ThiH or glycine by ThiO. These 3 components are combined by the enzyme ThiG, leading to the production of carboxy-thiazole phosphate (cTHZ-P). Finally, ThiE joins the pyrimidine (HMP-PP) and thiazole (cTHZ-P) moieties to generate thiamine monophosphate (TMP), which can be dephosphorylated by Arabidopsis thaliana phosphatase to form thiamine (FIG. 1).

In E. coli and anaerobic organisms, DHG is generated from tyrosine by the action of the enzyme ThiH using a complex radical-SAM mediated mechanism (D1 Broderick et al. 2014). The ThiH gene product of E. coli is sensitive to oxygen and its turnover value of 3 is very low for producing DHG (D1 Broderick et al. 2014). Other organisms, which are usually aerobic, use glycine instead of tyrosine as the substrate to produce DHG with a glycine oxidase (ThiO). In such organisms, ThiH is replaced by ThiO.

In this regard, the turnover values of the enzymes in B. subtilis (BsThiO) (D2 Boselli et al. 2007) and P. putida (PpThiO) (Equar et al. 2015) are orders of magnitude higher than those by ThiH from E. coli (Broderick et al. 2014) Interestingly, it has been suggested that ThiH can form a complex with ThiG for delivering DHG since it is a reactive compound owing to the aldehyde group (Leonardi et al 2003), whereas ThiF also interacts with ThiS (Lehmann et al. 2006), and then transfers the sulfur in an inactivated thio-carboxylated form to ThiG for making cTHZ-P in the presence of DXP and DHG.

In the case of the sulfur transfer sub-branch, IscS mobilizes the sulfur atom from cysteine to ThiI and the latter to ThiS (Taylor et al 1998; Martinez-Gomez et al 2011), whereas ThiF catalyzes the thio-carboxylation of ThiS (FIG. 2). Interestingly, E. coli contains all the following thiamine genes ThiF, ThiS, ThiI, IscS, ThiG and ThiH, but lacks ThiO. It is unclear what is the rate-limiting step in the THZ sub-branches. However, while sulfur transfer and sugar formation could be rate-limiting, the formation of DHG could also be improved by replacing ThiH with ThiO due to the less complex mechanism and higher turnover of the ThiO enzyme.

### SUMMARY

Against this background art, improvements have been provided in that ThiO enzymes from B. subtilis and P. putida can replace the function of the E. coli ThiH enzyme and that the levels of thiamine are greatly improved if the native ThiI gene is also co-expressed at a right level. Thus, the combination of replacing the ThiH enzyme from E. coli with ThiO enzymes from B. subtilis or P. putida and overexpressing ThiI at the right level is crucial for improving the formation of cTHZ-P, resulting in a significant improvement in the flux towards thiamine compared to the over-expression of ThiH alone or the ThiH and ThiI genes. This is illustrated in FIG. 2 and example 3 showing that ThiI co-expression increases the titer by >300% to ˜13 mg/L in strain BS06285 or BS06287 harbouring either a P. putida ThiO or B. subtilis ThiO enzyme, respectively.

Thus, overexpression of both ThiO and ThiI increased the flux to thiamine by approximately 30% in small-scale cultivations compared to a control with ThiH and ThiI. It is hypothesized that ThiO is not only a better enzyme than ThiH, but also that ThiI can work well with ThiOs from different organisms to increase the sulfur mobilization and formation of DHG and accordingly the production of thiamine. It is also found that the ThiI enzyme in a truncated form that contains a rhodanese domain, which is involved in sulfur mobilization to ThiS, effectively replaces the full-length native ThiI enzyme, further improving the production of thiamine. Hence, the results obtained herein show that the thiamine pathway can be improved significantly by replacing the radical SAM enzyme ThiH with a suitable combination and expression of ThiI and ThiO to enable the efficient production of thiamine from glucose or from added precursors.

Accordingly, in a first aspect, provided for herein is a genetically modified host cell having improved production of thiamine, wherein the host cell expresses one or more heterologous ThiO enzymes converting glycine into dehydroglycine (DHG) in the host cell, whereby the production of the thiamine in the genetically modified host cell is improved compared to an unmodified parent host cell.

In a second aspect, provided for herein is a genetically modified host cell having improved production of thiamine, wherein the host cell expresses one or more heterologous ThiI enzymes catalyzing the transfer of sulfur from IscS to the sulfur carrier protein ThiS in the host cell, whereby the production of the thiamine in the genetically modified host cell is improved compared to an unmodified parent host cell.

In a third aspect, provided for herein is a method for producing thiamine comprising:
a) culturing the cell culture at conditions allowing the host cells to produce the thiamine; and
b) optionally recovering and/or isolating the thiamine.

In a fourth aspect, provided for herein is a fermentation composition comprising the cell culture and the thiamine.

In a fifth aspect, provided for herein is a composition comprising the fermentation composition and one or more carriers, agents, adjuvants, additives and/or excipients.

In a final aspect, provided for herein is a method for treating a disease in a mammal, comprising administering a therapeutically effective amount of the composition to the mammal.

### DESCRIPTION OF DRAWINGS AND FIGURES

The figures included herein are illustrative and simplified for clarity, and they merely show details which are essential to the understanding of the invention, while other details may have been left out.

FIG. 1 shows the modules of the pathway for microbial production of thiamine in E. coli. In the pyrimidine branch (FIG. 1, module 1, left) ThiC (phosphomethylpyrimidine synthase) converts its substrate AIR (aminoimidazole ribotide) available from the purine pathway to HMP-P (hydroxymethylpyrimidine phosphate), which is then phosphorylated to HMP-PP (hydroxymethylpyrimidine diphosphate) by ThiD (hydroxymethylpyrimidine/phosphomethyl pyrimidine kinase). Since production of high levels of HMP-P is limiting due to a low turnover of ThiC, it is possible to overcome this limitation by co-expressing ThiD and externally feeding HMP, which is phosphorylated twice in a 2-step reaction to make HMP-PP (dotted arrow). In the thiazole (THZ) branch (FIG. 1, module 2, right) the main product is carboxy-thiazole phosphate (cTHZ-P), which is formed by the action of ThiG combining 3 molecules including sulfur from cysteine (by the action of IscS, ThiI, ThiS and ThiF), dehydroglycine (DHG) coming from either tyrosine (ThiH) in E. coli or glycine (ThiO) in various organisms and the sugar DXP (1-deoxy-D-xylulose 5-phosphate). THZ can also be fed to the cell factory with co-expression of ThiM (hydroxyethylthiazole kinase) to catalyse the phosphorylation of THZ to THZ-P (dotted arrow). ThiE (thiamine-phosphate synthase) condenses both HMP-PP and cTHZ-P to form thiamine monophosphate (TMP), which is dephosphorylated by A. thaliana phosphatase to produce thiamine (FIG. 1, module 3 (bottom)). Genes may be over-expressed from a plasmid (such as particularly ThiF, ThiS, ThiG, ThiH or ThiO, ThiI) or chromosome (such as particularly ThiD, ThiE ThiM and phosphatase). A single copy of the phosphatase is sufficient to generate approx. 98% thiamine, with the remaining being a mixture of TMP and thiamine diphosphate (TPP) based on LC-MS data (not shown).

FIG. 2 shows bar graphs of thiamine titre (mg/L), growth (OD) and OD-normalized titers (mg/L/OD) after 24 h batch cultivation in mMOPS (supplemented with 500 μM HMP) of E. coli strains expressing native thiamine genes ThiF, ThiS, ThiG and ThiH without (BS05850) or with (BS06232) ThiI co-expression. The other strains contain, instead of native ThiH, heterologous ThiOs from P. putida (BS06278) or B. subtilis (BS06280). Transformation of these strains with a plasmid encoding ThiI from E. coli yields the ThiI-containing strains BS06285 (P. putida ThiO) and BS06287 (B. subtilis ThiO). The bars show the median of 4 replicates and each dot represents one replicate.

FIG. 3 shows the four domains of ThiI from E. coli. The first domain (residues 1 to 63) contains an NFLD (N-terminal ferredoxin-like domain). The second one (residues 61-165) contains a THUMP (thiouridine synthases, methylases, and pseudouridine synthases) domain, both of which interact with tRNA. The third one (residues 189-360) belongs to an adenine nucleotide alpha-hydrolase (AANH) domain that is responsible for uridine adenylation. The fourth one (residues 404-482) has a rhodanese domain involved in the sulfur transfer to thiazole in the thiamine biosynthesis pathway. The ATP binding site is located between the second and third domains (residues 183-184) as well as within the third domain (residues 265-296).

FIG. 4 shows bar graphs of thiamine titre (mg/L) or growth (OD) after 24 h batch cultivation in mMOPS (supplemented with 500 μM HMP) of E. coli strains expressing in one synthetic operon the native thiamine genes ThiF, ThiS, ThiG, ThiI and P. putida ThiO in which ThiI (from E. coli) is co-expressed as the WT (BS07082) or truncated (BS07083) form. The truncated form corresponds to the rhodanese domain as explained in FIG. 3. The bars show the median of 4 replicates and the error bars the standard deviation.

FIG. 5 shows bench-top—fed-batch fermentation of strains over-expressing ThiI and either ThiO or ThiH (fed with HMP) at 31° C. and 15% DO. Strain BS06232 contains plasmids encoding ThiFSGH (pBS2819) and ThiI (pBS02896), whereas BS06285 has plasmids encoding ThiFSGO (pBS2919) and ThiI (pBS2896). Thiamine was quantified using the thiochrome assay (n=2).

FIG. 6 shows bench-top fed-batch fermentation of strains over-expressing ThiI and ThiO in the same operon (fed with HMP) at 31° C. and 15% DO. Strain BS06232 contains plasmids encoding ThiFSGH (pBS2819) and ThiI (pBS2896), while BS06375 has a plasmid encoding ThiFSGIO (pBS2967) alone. Thiamine was quantified using the thiochrome assay (n=2).

FIG. 7 shows bench-top fed-batch fermentation of strains with a native thiamine pathway over-expressing ThiI (fed with HMP) at 31° C. and 15% DO. Strain BS05897 contains a plasmid encoding ThiCEFSGH/ThiMD (pBS140), while BS06245 in addition to pBS140 contains the ThiI plasmid (pBS2896). Thiamine was quantified using the thiochrome assay (n=2).

FIG. 8 shows bench-top fed-batch fermentations of strains with native or synthetic thiamine pathways and without precursor addition (de novo) at 31° C. and 15% DO. Strain BS05897 contains a ThiCEFSGH operon (pBS140), whereas BS06468 has a ThiFSGIOCE operon (pBS3001). HMPs is the sum of precursors HMP, HMP-P and HMP-PP, whereas THZs is the sum of precursors THZ, THZ-P and cTHZ. Thiamine was quantified using the thiochrome assay, whereas all precursors were quantified via LC-MS.

FIG. 9 shows small-scale experiments with native (BS05897) or synthetic (BS06468) thiamine pathways without the addition of precursors (left) and with the addition of 500 μM HMP (middle) or 500 μM HMP+500 μM THZ (right). Strains were cultivated with 2 g/L glucose in minimal FIT medium (300 μL) plus yeast extract (1 g/L) at 37° C. for 24 h. Thiamine was quantified using the thiochrome assay (n=3). No difference in growth is observed for both strains with none or one precursor, whereas the native pathway strain showed a ˜20% higher OD compared to the synthetic pathway with both precursors.

FIG. 10 shows the performance of different modules of de novo strain BS06468 having a synthetic thiamine operon. Bench-top fed-batch fermentations without precursor addition (de novo) or with the addition of HMP (Module 2 of FIG. 1) or HMP plus THZ (Module 3 of FIG. 1) were performed at 31° C. and 15% DO. The precursors were added to enable a maximum yield of 15 mg thiamine per g glucose. Thiamine was quantified using the thiochrome assay, whereas thiamine monophosphate (TMP) and thiamine diphosphate (TPP) were quantified via LC-MS (n=1).

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

Provided for herein are genetically modified host cells having increased production of thiamine achieved by expressing one or more heterologous ThiO enzymes having higher turnover values for converting glycine into dehydroglycine (DHG) than the ThiH of E. coli, and one or more heterologous ThiI enzymes catalyzing the transfer of sulfur from IscS to the sulfur carrier protein ThiS thereby increasing the levels of active ThiS thiocarboxylated protein in the host cell, whereby the production of thiamine in the genetically modified host cell is improved compared to an unmodified parent host cell. Additionally, it was shown that a rhodanese domain of thiI is sufficient for producing thiamine.

### Definitions

Any EC numbers used herein refer to Enzyme Nomenclature 1992 from NC-IUBMB, Academic Press, San Diego, California, including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g., http://enzyme.expasy.org/. The term “PEP” as used herein refers to phosphoenol pyruvate.

The term “thiamine” as used herein may be used interchangeably about the compound thiamine as such, but also where appropriate thiamine mono phosphate (TMP) and/or thiamine pyrophosphate (TPP).

The term “TMP-phosphatase” as used herein refers to a thiamine monophosphate phosphatase dephosphorylating thiamine monophosphate to thiamine. It has been shown that for the bifunctional TH2 protein from Arabidopsis thaliana has this activity (see also WO2017103221).

The term “ThiK” as used herein refers to a thiamine kinase that catalyzes the phosphorylation of thiamine to thiamine-monophosphate (TMP).

The term “ThiL” as used herein refers to a thiamine-monophosphate kinase. It catalyzes the ATP-dependent phosphorylation of thiamine-monophosphate (TMP) to form thiamine-pyrophosphate (TPP), the active form of vitamin B1. It cannot use thiamine as a substrate. Is highly specific for ATP as a phosphate donor.

The term “ThiM” as used herein refers to a Hydroxyethylthiazole kinase that catalyzes the phosphorylation of the hydroxyl group of 4-methyl-5-beta-hydroxyethylthiazole.

The term “ThiD” as used herein refers to a Hydroxymethylpyrimidine or phosphomethylpyrimidine kinase that catalyzes the phosphorylation of hydroxymethylpyrimidine phosphate (HMP-P) to HMP-PP, and of HMP to HMP-P. ThiD shows no activity with pyridoxal, pyridoxamine or pyridoxine.

The term “ThiC” as used herein refers to a Phosphomethylpyrimidine synthase protein that catalyzes the synthesis of the hydroxymethylpyrimidine phosphate (HMP-P) moiety of thiamine from aminoimidazole ribotide (AIR) in a radical S-adenosyl-L-methionine (SAM)-dependent reaction.

The term “ThiE” as used herein refers to a thiamine-phosphate synthase protein that condenses 4-methyl-5-[2-(phosphonatooxy)ethyl]-1,3-thiazole-2-carboxylate (cTHZ-P) and 2-methyl-4-amino-5-hydroxymethyl pyrimidine pyrophosphate (HMP-PP) to form thiamine monophosphate (TMP).

The term “ThiF” as used herein refers to a sulfur carrier protein ThiS adenylyltransferase. ThiF catalyzes the adenylation of the carboxy terminus of ThiS and the subsequent displacement of AMP catalyzed by ThiI-persulfide to give a ThiS-ThiI acyl disulfide ThiS.

The term “ThiS” as used herein refers to a sulfur carrier protein in which a C-terminal thiocarboxylation occurs in 2 steps: First, it is acyl-adenlyated by ThiF and then thiocarboxylated by ThiI.

The term “ThiG” as used herein refers to a Thiazole synthase protein that catalyzes the rearrangement of 1-deoxy-D-xylulose 5-phosphate (DXP) to produce the thiazole phosphate moiety of thiamine. Sulfur is provided by the thiocarboxylate moiety of the carrier protein ThiS.

The term “ThiH” as used herein refers to a 2-iminoacetate synthase protein that catalyzes the radical SAM-mediated cleavage of tyrosine to 2-iminoacetate and 4-cresol.

The term “ThiI” as used herein refers to a tRNA sulfurtransferase protein that catalyzes the ATP-dependent transfer of sulfur to tRNA to produce 4-thiouridine in position 8 of tRNAs, which functions as a near-UV photosensor. Also catalyzes the transfer of sulfur to the sulfur carrier protein ThiS, forming ThiS-thiocarboxylate. This is a step in the synthesis of thiazole, in the thiamine biosynthesis pathway. The sulfur is donated as persulfide by IscS.

The term “Dxs protein” as used herein refers to a 1-deoxy-D-xylulose-5-phosphate synthase that catalyzes the acyloin condensation reaction between C atoms 2 and 3 of pyruvate and glyceraldehyde 3-phosphate to yield 1-deoxy-D-xylulose-5-phosphate (DXP).

The term “ThiO” as used herein refers to a glycine oxidase that catalyzes the FAD-dependent oxidative deamination of various amines and D-amino acids to yield the corresponding alpha-keto acids, ammonia/amine, and hydrogen peroxide including the formation of dehydroglycine from glycine. It is essential for thiamine biosynthesis in organisms that generally lack ThiH since the oxidation of glycine catalyzed by ThiO also generates the glycine imine intermediate (dehydroglycine) required for the biosynthesis of the thiazole ring of thiamine pyrophosphate.

The term “IscR” as used herein refers to HTH-type transcriptional regulator IscR that regulates the transcription of several operons and genes involved in the biogenesis of Fe-S clusters and Fe-S-containing proteins. Transcriptional repressor of the iscRSUA operon, which is involved in the assembly of Fe-S clusters into Fe-S proteins.

The term “host cell” refers to any cell type that is susceptible to transformation, transfection, transduction, or the like with a nucleic acid construct or expression vector comprising a polynucleotide of the present invention. The host cell encompasses any progeny of a parent cell that is not identical to the parent cell due to mutations that occur during replication.

The term “polynucleotide construct” refers to a polynucleotide, either single- or double-stranded, which is isolated from a naturally occurring gene or is modified to contain segments of nucleic acids in a manner that would not otherwise exist in nature, or which is synthetic, and which comprises a polynucleotide encoding a polypeptide and one or more control sequences.

The terms “nucleotide sequence” and “polynucleotide” are used herein interchangeably.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene or organism) or heterologous or foreign (i.e., from a different or organism) to the polynucleotide encoding the polypeptide. Control sequences include, but are not limited to leader sequences, polyadenylation sequences, pro-peptide coding sequences, promoter sequences, signal peptide coding sequences, translation terminator (stop) sequences and transcription terminator (stop) sequences. To be operational control sequences usually must include promoter sequences and transcriptional and translational stop signals. Control sequences may be provided with linkers for the purpose of introducing specific restriction sites facilitating ligation of the control sequences with a coding region of a polynucleotide encoding a polypeptide.

The term “expression vector” refers to a DNA molecule, either single- or double-stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “expression” includes any step involved in the production of a polypeptide including, but not limited to, transcription, post-transcriptional modification, translation, post-translational modification, and secretion.

The term “over-expression” includes a situation when one or more components of the cell may be present at a higher-than-normal cellular level (i.e., higher than the concentration known to usually be present in the cell type exhibiting the gene and/or protein complex of interest). For example, the gene encoding a protein may begin to be overexpressed, or may be amplified (i. e., its gene copy number may be increased) in certain cells, leading to an increased number of component molecules within these cells.

The terms “heterologous” or “recombinant” or “genetically modified” and their grammatical equivalents as used herein interchangeably refer to entities “derived from a different species or cell”. For example, a heterologous or recombinant polynucleotide gene is a gene in a host cell not naturally containing that gene, i.e., the gene is from a different species or cell type than the host cell. The terms as used herein about microbial host cells refer to microbial host cells comprising and expressing heterologous or recombinant polynucleotide genes.

The term “metabolic pathway” as used herein is intended to mean two or more enzymes acting sequentially in a live cell to convert chemical substrate(s) into chemical product(s). Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds such as proteins for example enzymes (co-enzymes). A CPR that reduces certain cytochrome P450 enzymes is an example of an enzymatic co-factor. The term “operative biosynthetic metabolic pathway” refers to a metabolic pathway that occurs in a live recombinant host, as described herein.

The term “in vivo”, as used herein refers to within a living cell or organism, including, for example, animal, a plant or a microorganism.

The term “in vitro”, as used herein refers to outside a living cell or organism, including, without limitation, for example, in a microwell plate, a tube, a flask, a beaker, a tank, a reactor and the like.

The term “substrate” or “precursor”, as used herein refers to any compound that can be converted into a different compound. For example, Air can be a substrate for Phosphomethylpyrimidine synthase and can be converted into thiamine into hydroxymethylpyrimidine phosphate (HMP-P), a precursor of thiamine. For clarity, substrates and/or precursors include both compounds generated in situ by an enzymatic reaction in a cell or exogenously provided compounds, such as exogenously provided organic molecules which the host cell can metabolize into a desired compound.

The term “endogenous” or “native” as used herein refers to a gene or a polypeptide in a host cell which originates from the same host cell.

The term “deletion” as used herein refers to the manipulation of a gene so that it is no longer expressed in a host cell.

The term “attenuation” as used herein refers to the manipulation of a gene or any of the machinery participating in the expression of the gene so that the expression of the gene is reduced as compared to expression without the manipulation.

The terms “substantially” or “approximately” or “about”, as used herein refer to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value, the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further, the wording X, Y and/or Z is intended to mean X, Y and Z alone or any combination of X, Y, and Z.

The term “isolated” as used herein about a compound, refers to any compound, by means of human intervention, has been put in a form or environment that differs from the form or environment in which it is found in nature. Isolated compounds include but is no limited to compounds disclosed herein for which the ratio of the compounds relative to other constituents with which they are associated in nature is increased or decreased. In an important embodiment, the amount of compound is increased relative to other constituents with which the compound is associated in nature. In an embodiment, the compound disclosed herein may be isolated into a pure or substantially pure form. In this context, a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment, the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “% identity” is used herein about the relatedness between two amino acid sequences or between two nucleotide sequences. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. “% identity” as used herein about amino acid sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

$$\frac{identical amino acid residues}{Length of alignment - total number of gaps in alignment} \times 100$$

The term “% identity” as used herein about nucleotide sequences refers to the degree of identity in percent between two deoxyribonucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

$$\frac{identical amino acid residues}{Length of alignment - total number of gaps in alignment} \times 100$$

Other examples of algorithms that are suitable for determining percent sequence identity and sequence similarity include the BLAST and BLAST 2.0 algorithms, which are described in Altschul, et al. (1977) Nuc. Acids Res. 25: 3389-3402 and Altschul, et al. (1990) J.Mol. Biol. 215: 403-410, or the WU-BLAST-2 program (Altschul et al., Meth. Enzymol., 266: 460-480 (1996).

The protein sequences of the present invention can further be used as a “query sequence” to perform a search against sequence databases, for example, to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “mature polypeptide” or “mature enzyme” as used herein refers to a polypeptide in its final active form following translation and any post-translational modifications, such as N-terminal processing, C-terminal truncation, glycosylation, phosphorylation, etc. It is known in the art that a host cell may produce a mixture of two or more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The terms “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein to refer to one or to more than one (i.e., to one or at least one) of the grammatical object of the article. By way of example, “an element” may mean one element or more than one element.

Terms like “preferably”, “commonly”, “particularly”, and “typically” are not utilized herein to limit the scope of the item's invention or to imply that certain features are critical, essential, or even important to the structure or function of the item invention. Rather, these terms are merely intended to highlight alternative or additional features that can or cannot be utilized in a particular embodiment of the present invention.

The term “cell culture” as used herein refers to a culture medium comprising a plurality of host cells disclosed herein. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., one or more of (i) trace metals; (ii) vitamins; (iii) salts (such as salts of phosphate, magnesium, potassium, zinc, iron); (iv) nitrogen sources (such as YNB, ammonium sulphate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418 and hygromycin B).

The term “radical SAM” as used herein refers to a superfamily of enzymes that use a [4Fe-4S]⁺ cluster to reductively cleave S-adenosyl-L-methionine (SAM) to generate a radical, usually a 5′-deoxyadenosyl radical, as a critical intermediate. The vast majority of known radical SAM enzymes have a cysteine-rich motif that matches or resembles CxxxCxxC.

### Genetically Modified Host Cells

The genetically modified host cells having increased production of thiamine, provided for herein, expresses in some embodiments one or more heterologous ThiO enzymes converting glycine into dehydroglycine (DHG) in the host cell, whereby the production of the thiamine in the genetically modified host cell is improved compared to an unmodified parent host cell. The genetically modified host cells having increased production of thiamine, provided for herein, expresses in other one or more heterologous ThiI enzymes catalyzing the transfer of sulfur from IscS to the sulfur carrier protein ThiS in the host cell, whereby the production of the thiamine in the genetically modified host cell is improved compared to an unmodified parent host cell.

The increase in the genetically modified host cell's capacity to produce thiamine can in some embodiments be at least 50%, such as at least 100%, such as least 150%, such as at least 200%. In other embodiments the genetical modification of the host cells having increased production of thiamine, provided for herein, include one or more mutations in native polynucleotide constructs encoding one or more proteins selected from protein cAMP receptor protein (CRP), carbohydrate repression resistance protein (CRR) and adenylate cyclase protein (CyaA) as described in patent application PCT/EP2022/069711.

In some embodiments, the genetically modified host cell having improved production of thiamine, is a cell wherein the one or more heterologous ThiO enzymes is a bacterial ThiO enzyme. In another embodiment, the genetically modified host cell having improved production of thiamine, is a cell wherein the one or more heterologous ThiI enzymes is a bacterial ThiI enzyme. In a further embodiment, the genetically modified host cell having improved production of thiamine is a cell wherein the one or more heterologous bacterial ThiO enzymes is a BsThiO enzyme from B. subtilis and/or a PpThiO enzyme from P. putida. In other embodiments, the heterologous bacterial ThiO enzymes from B. subtilis and/or PpThiO enzyme from P. putida is at least 70%, such as at least 80%, such as at least 90%, such as at least 95%, such as at least 96%, such as at least 97%, such as at least 98%, such as at least 99%, such as 100% identical to the ThiO enzyme comprised in SEQ ID NO 27 and SEQ ID NO 29. In one embodiment, the bacterial ThiI enzyme is an E. coli ThiI enzyme having SEQ ID NO: 23. In another embodiment the bacterial ThiO gene is a BsThiO gene from B. subtilis having SEQ ID NO: 28 and/or a PpThiO gene from P. putida having SEQ ID NO: 30.

In some embodiments, the gene encoding:
a) the ThiO enzyme is at least 50%, such as at least 60%, such as at least 70%, such as at least 80%, such as at least 90%, such as at least 95%, such as at least 96%, such as at least 97%, such as at least 98%, such as at least 99%, such as 100% identical to the enzyme comprised in SEQ ID NO: 27 or 29 or genomic DNA thereof encoding the ThiO enzyme comprised in SEQ ID NO: 28 or 30;
b) the ThiI enzyme is least 50%, such as at least 60%, such as at least 70%, such as at least 80%, such as at least 90%, such as at least 95%, such as at least 96%, such as at least 97%, such as at least 98%, such as at least 99%, such as 100% identical to the enzyme comprised in SEQ ID NO: 23 or genomic DNA thereof encoding the ThiI enzyme comprised in SEQ ID NO: 24.

In a still further embodiment, the:
a) BsThiO gene is at least 70%, such as at least 80%, such as at least 90%, such as at least 95%, such as at least 96%, such as at least 97%, such as at least 98%, such as at least 99%, such as 100% identical to the BsThiO gene comprised in SEQ ID NO: 28;
b) PpThiO gene is at least 70%, such as at least 80%, such as at least 90%, such as at least 95%, such as at least 96%, such as at least 97%, such as at least 98%, such as at least 99%, such as 100% identical to the PpThiO gene comprised in SEQ ID NO: 30.

In one embodiment, the ThiI gene is a truncated ThiI gene comprising a rhodanese domain that transfers the sulfur from IscS as a persulfide to the sulfur carrier protein ThiS, forming ThiS-thiocarboxylate in the thiamine biosynthesis pathway. In a further embodiment, the rhodanese domain is at least 70%, such as at least 80%, such as at least 90%, such as at least 95%, such as at least 96%, such as at least 97%, such as at least 98%, such as at least 99%, such as 100% identical to the rhodanese domain comprised in SEQ ID NO: 34. In another embodiment, the ThiI enzyme is a truncated ThiI enzyme comprising a rhodanese domain that transfers the sulfur from IscS as a persulfide to the sulfur carrier protein ThiS, forming ThiS-thiocarboxylate in the thiamine biosynthesis pathway. In a further embodiment, the rhodanese domain is at least 70%, such as at least 80%, such as at least 90%, such as at least 95%, such as at least 96%, such as at least 97%, such as at least 98%, such as at least 99%, such as 100% identical to the rhodanese domain comprised in SEQ ID NO: 33.

In some embodiments, the genetically modified host cell, further comprises an operative metabolic pathway comprising one or more native or heterologous pathway elements producing the thiamine. In one embodiment, the one or more pathway elements comprise one or more radical SAM enzymes. In another embodiment, the one or more pathway elements are selected from:
a) one or more phosphate synthase enzymes selected from phosphomethylpyrimidine synthase (ThiC); that catalyzes the synthesis of the hydroxymethylpyrimidine phosphate (HMP-P) moiety of thiamine from aminoimidazole ribotide (AIR) in a radical S-adenosyl-L-methionine (SAM)-dependent reaction;
b) a hydroxymethylpyrimidine/phosphomethylpyrimidine kinase (ThiD) that catalyzes the phosphorylation of hydroxymethylpyrimidine phosphate (HMP-P) to HMP-PP, and of HMP to HMP-P;
c) an adenylyltransferase (ThiF) that catalyzes the adenylation of the carboxy terminus of the sulfur carrier protein ThiS and the subsequent displacement of AMP catalyzed by ThiI-persulfide to give a ThiS-ThiI acyl disulfide ThiS;
d) a sulfur carrier protein (ThiS) in which its C-terminal thiocarboxylation occurs in 2 steps: First, it is acyl-adenlyated by ThiF and then thiocarboxylated by ThiI;
e) a 2-iminoacetate synthase (ThiH) converting that catalyzes the radical-mediated cleavage of tyrosine to 2-iminoacetate and 4-cresol;
f) a thiazole synthase (ThiG) that catalyzes the rearrangement of 1-deoxy-D-xylulose 5-phosphate (DXP) to produce the thiazole phosphate moiety of thiamine;
g) a hydroxyethylthiazole kinase (ThiM) that catalyzes the rearrangement of 1-deoxy-D-xylulose 5-phosphate (DXP) to produce the thiazole phosphate moiety of thiamine;
h) a thiamine monophosphate (TMP) phosphatase that dephosphorylate thiamine monophosphate to thiamine;
i) a thiamine kinase (ThiK) that catalyzes the phosphorylation of thiamine to thiamine phosphate;
j) a thiamine-monophosphate kinase (ThiL) which catalyzes the ATP-dependent phosphorylation of thiamine-monophosphate (TMP) to form thiamine-pyrophosphate (TPP);
k) a thiamine-phosphate synthase (ThiE) that condenses 4-methyl-5-[2-(phosphonatooxy)ethyl]-1,3-thiazole-2-carboxylate (cTHZ-P) and 2-methyl-4-amino-5-hydroxymethyl pyrimidine pyrophosphate (HMP-PP) to form thiamine monophosphate (TMP);
l) an HTH-type transcriptional regulator (IscR) that regulates the transcription of several operons and genes involved in the biogenesis of Fe-S clusters and Fe-S-containing proteins.

In a further embodiment, the one or more pathway elements are encoded by one or more genes selected from the group of:
a) ThiC that has at least 70%, such at least 75%, such as at least 80%, such as at least 90%, such as at least 95%, such as at least 99%, such as 100% identity to the ThiC comprised in SEQ ID NO: 2;
b) ThiD that has at least 70%, such at least 75%, such as at least 80%, such as at least 90%, such as at least 95%, such as at least 99%, such as 100% identity to the ThiD comprised in SEQ ID NO: 4;
c) ThiF that has at least 70%, such at least 75%, such as at least 80%, such as at least 90%, such as at least 95%, such as at least 99%, such as 100% identity to the ThiF comprised in SEQ ID NO: 6;
d) ThiS has at least 70%, such at least 75%, such as at least 80%, such as at least 90%, such as at least 95%, such as at least 99%, such as 100% identity to the ThiS comprised in SEQ ID NO: 8;
e) ThiH that has at least 70%, such at least 75%, such as at least 80%, such as at least 90%, such as at least 95%, such as at least 99%, such as 100% identity to the ThiH comprised in SEQ ID NO: 10;
f) ThiG that has at least 70%, such at least 75%, such as at least 80%, such as at least 90%, such as at least 95%, such as at least 99%, such as 100% identity to the ThiG comprised in SEQ ID NO: 12;
g) ThiM that has at least 70%, such at least 75%, such as at least 80%, such as at least 90%, such as at least 95%, such as at least 99%, such as 100% identity to the ThiM comprised in SEQ ID NO: 14;
h) TMP phosphatase that has at least 70%, such at least 75%, such as at least 80%, such as at least 90%, such as at least 95%, such as at least 99%, such as 100% identity to the TMP phosphatase comprised in SEQ ID NO: 16;
i) ThiK that has at least 70%, such at least 75%, such as at least 80%, such as at least 90%, such as at least 95%, such as at least 99%, such as 100% identity to the ThiK comprised in SEQ ID NO: 18;
j) ThiL that has at least 70%, such at least 75%, such as at least 80%, such as at least 90%, such as at least 95%, such as at least 99%, such as 100% identity to the ThiL comprised in SEQ ID NO: 20;
k) ThiE that has at least 70%, such at least 75%, such as at least 80%, such as at least 90%, such as at least 95%, such as at least 99%, such as 100% identity to the ThiE comprised in SEQ ID NO: 22;
l) IscR that has at least 70%, such at least 75%, such as at least 80%, such as at least 90%, such as at least 95%, such as at least 99%, such as 100% identity to the IscR comprised in SEQ ID NO: 32.

In some embodiments, the one or more pathway elements are encoded by one or more genes selected from the group of:
a) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 2 or genomic DNA thereof encoding the ThiC enzyme comprised in SEQ ID NO: 1;
b) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 4 or genomic DNA thereof encoding the ThiD enzyme comprised in SEQ ID NO:3;
c) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 6 or genomic DNA thereof encoding the ThiF enzyme comprised in SEQ ID NO: 5;
d) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 8 or genomic DNA thereof encoding the ThiS enzyme comprised in SEQ ID NO: 7;
e) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 10 or genomic DNA thereof encoding the ThiH enzyme comprised in SEQ ID NO: 9;
f) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 12 or genomic DNA thereof encoding the ThiG enzyme comprised in SEQ ID NO: 11;
g) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 14 or genomic DNA thereof encoding the ThiM enzyme comprised in SEQ ID NO: 13;
h) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 16 or genomic DNA thereof encoding the TMP enzyme phosphatase comprised in SEQ ID NO: 15;
i) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 18, or genomic DNA thereof encoding the ThiK enzyme comprised in SEQ ID NO: 17;
j) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 20, or genomic DNA thereof encoding the ThiL enzyme comprised in SEQ ID NO: 19;
k) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 22, or genomic DNA thereof encoding the ThiE enzyme comprised in SEQ ID NO: 23;
l) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 24, or genomic DNA thereof encoding the ThiI enzyme comprised in SEQ ID NO: 23;
m) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 32, or genomic DNA thereof encoding the IscR enzyme comprised in SEQ ID NO: 31.

In some embodiments, the genetically modified host cell is a cell wherein the endogenous native ThiH gene is deleted, disrupted and/or attenuated. In a further embodiment, the deletion, disruption and/or attenuation is caused by introducing a deletion through complete removal of the gene or a translational knockout by introducing one or more stop codons or frameshift mutations preventing expression of an active peptide. In another embodiment, the deletion, disruption and/or attenuation comprises a translational knockout or a frameshift mutation. In another embodiment, the deletion, disruption and/or attenuation comprises a translational knockout or a frameshift mutation. In a further embodiment, the deletion, disruption and/or attenuation is a point mutation in a promoter for the protein-encoding sequence, in the RBS region and/or in a protein-encoding sequence.

In some embodiments, one or more genes and/or polypeptides of the pathway for the thiamine are heterologous to the host cell. In another embodiment, the host cell further comprises at least 2 copies of one or more genes and/or polypeptides of the pathway for the thiamine.

In a further embodiment, the host cell further comprises a transporter molecule facilitating transport of a precursor for or a product of the pathway for the thiamine. In one embodiment, the host cell is further genetically modified to provide an increased amount of a substrate in the pathway for the thiamine. In another embodiment, the host cell is further genetically modified to exhibit increased tolerance towards one or more substrates, intermediates, or products in the pathway for the thiamine. In further embodiment, the one or more additional native or endogenous genes of the host cell are deleted, disrupted and/or attenuated. In a still further embodiment, one or more genes in the pathway for the thiamine are overexpressed. In another embodiment, the host cell is further genetically modified to provide an increased amount of a substrate in the pathway for the thiamine.

### Host Cells.

The host cell disclosed herein may be any host cell suitable for hosting and expressing the [pathway for the thiamine. Such cell may be a prokaryotic or eukaryotic cell. Suitable prokaryotic host cells can be of a genus selected from Escherichia, Bacillus, Brevibacterium, Burkholderia, Campylobacter, Corynebacterium, Serratia, Lactobacillus, Lactococcus, Acinetobacter, Acetobacter or Pseudomonas. Particularly useful prokaryotic host cells are of the genus Escherichia, Corynebacterium, Bacillus, Serratia, or Pseudomonas, such as the species Escherichia coli, Corynebacterium glutamicum, B. subtilis, Serratia marcescens, P. putida and/or Pseudomonas mutabilis. Useful eukaryotic host cells include mammalian, insect, plant, fungal or archaeal cells. Among the eukaryotic host cells fungal cells of the genus Saccharomyces, Kluveromyces, Candida, Pichia, Debaromyces, Hansenula, Yarrowia, Zygosaccharomyces, Schizosaccharomyces and Ashbya are particularly useful, such as the species Kluyveromyces lactis, Saccharomyces carlsbergensis, Saccharomyces cerevisiae, Saccharomyces diastaticus, Saccharomyces douglasii, Saccharomyces kluyveri, Saccharomyces bensis, Saccharomyces oviformis, Yarrowia lipolytica, Pichia pastoris or Ashbya gossypii.

### Polynucleotide Constructs and Expression Vectors

Also provided for herein are polynucleotide constructs harboring gene(s) encoding native or mutated ThiC, ThiD, ThiF, ThiS, ThiH, ThiG, ThiM, ThiE, TMP phosphatase, ThiK, ThiL, ThiI, IscR and ThiO genes operably linked to one or more control sequences, said polynucleotide constructs comprising mutations to delete, disrupt, and/or an attenuate the gene transcription or translation or the activity and/or function of the encoded genes. The control sequences direct the expression of the encoded ThiC, ThiD, ThiF, ThiS, ThiH, ThiG, ThiM, ThiE, TMP phosphatase, ThiK, ThiL, ThiI, IscR or ThiO genes in the host cell harboring the polynucleotide construct. Conditions for the expression should be compatible with the control sequences. The control sequence may be heterologous or native to the gene(s) encoding the ThiC, ThiD, ThiF, ThiS, ThiH, ThiG, ThiM, ThiE, TMP phosphatase, ThiK, ThiL, ThiI, IscR or ThiO genes and/or to the host cell. In some embodiments, both the control sequence and the gene(s) encoding ThiC, ThiD, ThiF, ThiS, ThiH, ThiG, ThiM, ThiE, TMP phosphatase, ThiK, ThiL, ThiI, IscR or ThiO are heterologous to the host cell and optionally also to each other. In one embodiment the polynucleotide construct is an expression vector, comprising the gene(s) encoding the ThiC, ThiD, ThiF, ThiS, ThiH, ThiG, ThiM, ThiE, TMP phosphatase, ThiK, ThiL, ThiI, IscR or ThiO operably linked to the one or more control sequences.

Polynucleotides may be manipulated in a variety of ways to modify the expression of the ThiC, ThiD, ThiF, ThiS, ThiH, ThiG, ThiM, ThiE, TMP phosphatase, ThiK, ThiL, ThiI, IscR or ThiO. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may also be an inducible promoter. Selecting a suitable promoter for expression in yeast is well-known and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used. The control sequence may also be an mRNA stabilizer region downstream of a promoter and upstream of the coding sequence of a gene which increases the expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

It may also be desirable to add regulatory sequences that regulate the expression of the ThiC, ThiD, ThiF, ThiS, ThiH, ThiG, ThiM, ThiE, TMP phosphatase, ThiK, ThiL, ThiI, IscR or ThiO genes relative to the growth of the host cell. Examples of regulatory systems are those that cause expression of the gene to be turned on or off in response to a chemical or physical stimulus, including the presence of a regulatory compound.

Various nucleotide sequences in addition to the polynucleotide construct disclosed herein may be joined together to produce a recombinant expression vector, which may include one or more convenient restriction sites to allow for insertion or substitution of the polynucleotide sequence encoding the ThiC, ThiD, ThiF, ThiS, ThiH, ThiG, ThiM, ThiE, TMP phosphatase, ThiK, ThiL, ThiI, IscR or ThiO genes at such sites. The recombinant expression vector may be any vector (e.g., a plasmid or virus or chromosomal) that can be conveniently subjected to recombinant DNA procedures and can bring about expression of the ThiC, ThiD, ThiF, ThiS, ThiH, ThiG, ThiM, ThiE, TMP phosphatase, ThiK, ThiL, ThiI, IscR or ThiO genes. The choice of the vector will typically depend on the compatibility of the vector with the host cell into which the vector is to be introduced. The vector may be an autonomously replicating vector, i.e., a vector that exists as an extrachromosomal entity, the replication of which is independent of chromosomal replication, e.g., a plasmid (linear or closed circular plasmid), an extrachromosomal element, a mini-chromosome, or an artificial chromosome. The vector may contain any means for assuring self-replication. Alternatively, the vector may, when introduced into the host cell, integrate into the genome, and replicate together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like.

The vector may further contain element(s) that permit integration of the vector into genome (being a vector in itself) of the host cell or permits autonomous replication of the vector in the cell independent of the genome. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” refers to a polynucleotide that enables a plasmid or vector to replicate in vivo.

As mentioned, supra, more than one copy of a gene encoding pathway elements for thiamine may be inserted into a host cell to increase the production of thiamine. An increase in the gene copy number can be obtained by integrating one or more additional copies of a gene into the host cell genome or by including an amplifiable selectable marker gene with the gene so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present disclosure are well known to one skilled in the art (see, e.g., Sambrook et al., 1989, supra).

In a separate embodiment, the host cell comprises the polynucleotide constructs and/or vectors as disclosed herein.

### Cultures

Further provided for herein are cell cultures comprising the genetically modified host cells disclosed herein and a growth medium. Suitable growth mediums for relevant prokaryotic or eukaryotic host cells are widely known in the art.

### Methods of Producing Compounds.

This disclosure also describes a method for producing thiamine comprising
a) culturing the cell culture disclosed herein at conditions allowing the host cells to produce the thiamine; and
b) optionally recovering and/or isolating the thiamine.

The cell culture can be cultivated in a nutrient medium and at conditions suitable for the production of the thiamine disclosed herein and/or for propagating cell count using methods known in the art. For example, the culture may be cultivated by shake flask cultivation, or small-scale or large-scale fermentation (including continuous, batch, fed-batch, or solid state fermentations) in a laboratory or industrial fermenters in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g., from catalogues of the American Type Culture Collection). The selection of the appropriate medium may be based on the choice of host cell and/or based on the regulatory requirements for the host cell. Such media are available in the art. The medium may, if desired, contain additional components favouring the transformed expression hosts over other potentially contaminating microorganisms. Accordingly, in an embodiment a suitable nutrient medium can include one or more of (i) trace metals; (ii) vitamins; (iii) salts (such as salts of phosphate, magnesium, potassium, zinc, iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418 and hygromycin B).

The cultivation of the host cell may be performed over a period of time from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions are a temperature in the range of from about 25° C. to about 55° C. and at a pH of from about 3 to about 9. The appropriate conditions are usually selected based on the choice of host cell.

Accordingly, in some embodiments the method disclosed herein comprises one or more elements selected from:
a) culturing the cell culture under aerobic or anaerobic conditions
b) cultivating the host cells under mixing;
c) cultivating the host cells at a temperature of between 25° C. to 50° C.;
d) cultivating the host cells at a pH of between 3-9; and
e) cultivating the host cells for between 10 hours to 120 days.

The cell culture disclosed herein may be recovered and or isolated using methods known in the art. For example, the compound(s) may be recovered from the nutrient medium by conventional procedures including, but not limited to, centrifugation, filtration, spray-drying, or lyophilization. In a particular embodiment the method includes a recovery and/or isolation step comprising separating a liquid phase of the cell culture from a solid phase of the cell culture to obtain a supernatant comprising the thiamine and subjecting the supernatant to one or more steps selected from:
a) contacting the supernatant with one or more adsorbent resins to obtain at least a portion of the produced thiamine, then optionally recovering the thiamine from the resin in a concentrated solution prior to isolation of the thiamine by crystallisation or solvent evaporation;
b) contacting the supernatant with one or more ion exchange or reversed-phase chromatography columns to obtain at least a portion of the thiamine, then optionally recovering the thiamine from the resin in a concentrated solution prior to isolation of the thiamine by crystallisation or solvent evaporation;
c) extracting the thiamine from the supernatant, such as by liquid-liquid extraction into an immiscible solvent, then optionally isolating the thiamine by crystallisation or solvent evaporation; and
   thereby recovering and/or isolating the thiamine.

The thiamine yield provided by the method disclosed herein is typically higher than when producing thiamine by methods employing host cells without reduced CRP-cAMP complex formation and/or increased degradation and/or decreased binding of cAMP, in some embodiments at least 10% higher such as at least 50%, such as at least 100%, such as least 150%, such as at least 200% higher.

In one embodiment the thiamine yield and/or titer obtained by the method disclosed herein is from at least 50 mg/L to 800 mg/L thiamine. In other embodiments thiamine yield and/or titer obtained by the method is at least 400 mg/L, such as at least 800 mg/L, such as at least 1000 mg/L, such as at least 1500 mg/L, such as at least 2000 mg/L, such as at least 3000 mg/L, such as at least 5000 mg/L, such as at least 10000 mg/L, such as at least 15000 mg/L, such as at least 20000 mg/L.

The method disclosed herein may further comprise one or more steps of mixing the thiamine with one or more carriers, agents, adjuvants, additives and/or excipients, optionally pharmaceutical grade carriers, agents, adjuvants, additives and/or excipients.

The method disclosed herein may further comprise one or more in vitro steps in the process of producing the thiamine. It may also comprise one or more in vivo steps performed in another cell than the host cell disclosed herein. For example, precursors for and/or intermediates in the pathway for the thiamine may be produced in another cell and isolated therefrom and then fed to a cell culture disclosed herein for conversion into the thiamine. Accordingly, in one embodiment the method disclosed herein further comprises feeding one or more exogenous thiamine precursors to the host cell culture. In a preferred embodiment the feeding one or more exogenous thiamine precursors to the host cell culture comprises feeding THZ, HMP or THZ to the cell culture.

### Fermentation Composition

This disclosure also describes a fermentation composition comprising the cell culture disclosed herein and the thiamine—either comprised in the cells or in the medium. In the fermentation composition the genetically modified host cells may be wholly or partially lysed and/or disintegrated. In some embodiments at least 50%, such as at least 75%, such as at least 95%, such as at least 99% of the genetically modified host cells in the fermentation composition are lysed and/or disintegrated. Further, in the fermentation composition disclosed herein at least 50%, such as at least 75%, such as at least 95%, such as at least 99% of solid cellular material may have been separated and/or removed from a liquid phase of the fermentation composition.

The fermentation composition may further comprise one or more compounds of
a) precursor or products of the operative metabolic pathway producing the thiamine;
b) supplemental nutrients comprising; and
   wherein the concentration of the thiamine is at least 1 mg/L composition. In particular the fermentation composition can comprise a concentration of thiamine of at least 5 mg/kg, such as at least 10 mg/kg, such as at least 20 mg/kg, such as at least 50 mg/kg, such as at least 100 mg/kg, such as at least 500 mg/kg, such as at least 1000 mg/kg, such as at least 5000 mg/kg, such as at least 10000 mg/kg, such as at least 50000 mg/kg.

Suitable supplemental nutrients can include one or more of (i) trace metals; (ii) vitamins; (iii) salts (such as salts of phosphate, magnesium, potassium, zinc, and iron); (iv) nitrogen sources (such as YNB, ammonium sulfate, urea, yeast extracts, ammonium nitrate, ammonium chloride, malt extract, peptone and/or amino acids); (v) carbon source (such as dextrose, sucrose, glycerol, glucose, maltose, molasses, starch, cellulose, xylan, pectin, lignocellolytic biomass hydrolysate, and/or acetate); (vi) nucleobases; (vii) aminoglycosides; and/or (viii) antibiotics (such as G418, hygromycin B, spectinomycin and/or Kanamycin).

### Compositions and Use

This disclosure also describes a composition comprising the fermentation composition disclosed herein and one or more carriers, agents, adjuvants, additives and/or excipients. Suitable carriers, agents, adjuvants, additives and/or excipients include formulation additives, stabilising agents, fillers, and the like. The composition and the one or more carriers, agents, adjuvants, additives and/or excipients can suitably be formulated into a dry solid form, e.g., by using methods known in the art, such as spray drying, spray cooling, lyophilization, flash freezing, granulation, microgranulation, encapsulation or microencapsulation. The composition and the one or more carriers, agents, adjuvants, additives and/or excipients can also be formulated into a liquid stabilized form using methods known in the art, such as adding to the fermentation composition one or more stabilizers such as sugars and/or polyols (e.g., sugar alcohols) and/or organic acids (e.g., lactic acid).

The composition disclosed herein may be further refined into a pharmaceutical preparation, a dietary supplement, a cosmetic, a food preparation, a feed preparation and/or an analytical or diagnostic reagent optionally using one or more steps of the methods described herein for producing the thiamine including mixing the thiamine with one or more pharmaceutical grade carriers, agents, adjuvants, additives and/or excipients. In one embodiment, the pharmaceutical composition is a pharmaceutical preparation obtainable from the method disclosed herein. The pharmaceutical preparation may be a dry preparation, optionally in the form of a powder, tablet, capsule, hard chewable and or soft lozenge or a gum. Alternatively, the pharmaceutical preparation may in form of a liquid pharmaceutical solution. Such pharmaceutical preparations may be used as a medicament in a method for treating and/or relieving a disease and/or medical condition, in particular in a mammal, in particular for use in the treatment of a nutritional deficiency. Accordingly, this disclosure further describes a method for preventing, treating and/or relieving a disease and/or medical condition comprising administering a therapeutically effective amount of the pharmaceutical composition disclosed herein to a mammal in need of treatment and/or relief. Diseases and/or medical conditions treatable or relievable by the pharmaceutical composition include but are not limited to diseases and/or medical conditions associated with lacking or insufficient bodily intake of thiamine. The pharmaceutical preparation can be administered parenterally, such as topically, epicutaneously, sublingually, buccally, nasally, intradermally, intravenously, and/or intramuscularly. The pharmaceutical composition can also be administered enterally via the gastrointestinal tract.

This disclosure also describes a kit of parts comprising:
a) the genetically modified host cell as described herein; and/or
b) instructions for use of the genetically modified host cell; and/or
c) the nucleic acid construct as described herein; and/or
d) instructions for use of the nucleic acid construct; and/or
e) a host cell which can be genetically modified using the methods described herein.

In some embodiments, the kit comprises a genetically modified cell capable of producing thiamine, wherein the genetically modified cell expresses pathway enzymes producing the thiamine. In one embodiment the genetically modified cell expresses thiamine synthase (ThiC) and optionally one or more thiamine pathway enzymes or factors selected from ThiD, ThiF, ThiS, ThiH, ThiG, ThiM, ThiE, TMP phosphatase, ThiK, ThiL, ThiI, IscR and ThiO.

The present application contains a Sequence Listing prepared in PatentIn included below but also submitted electronically in ST26 format which is hereby incorporated by reference in its entirety.

### SEQUENCE LISTING

The present application contains a Sequence Listing prepared in PatentIn included below but also submitted electronically in ST26 format which is hereby incorporated by reference in its entirety.

SEQ ID NO: 1: Protein sequence of ThiC
SEQ ID NO: 2: DNA sequence encoding ThiC
SEQ ID NO: 3: Protein sequence of ThiD
SEQ ID NO: 4: DNA sequence encoding ThiD
SEQ ID NO: 5: Protein sequence of ThiF
SEQ ID NO: 6: DNA sequence encoding ThiF
SEQ ID NO: 7: Protein sequence of ThiS
SEQ ID NO: 8: DNA sequence encoding ThiS
SEQ ID NO: 9: Protein sequence of ThiH
SEQ ID NO: 10: DNA sequence encoding ThiH
SEQ ID NO: 11: Protein sequence of ThiG
SEQ ID NO: 12: DNA sequence encoding ThiG
SEQ ID NO: 13: Protein sequence of ThiM
SEQ ID NO: 14: DNA sequence encoding ThiM
SEQ ID NO: 15: Protein sequence of TMP Phosphatase
SEQ ID NO: 16: DNA sequence encoding TMP Phosphatase
SEQ ID NO: 17: Protein sequence of Thik
SEQ ID NO: 18: DNA sequence encoding Thik
SEQ ID NO: 19: Protein sequence of ThiL
SEQ ID NO: 20: DNA sequence encoding ThiL
SEQ ID NO: 21: Protein sequence of ThiE
SEQ ID NO: 22: DNA sequence encoding ThiE
SEQ ID NO: 23: Protein sequence of ThiI WT
SEQ ID NO: 24: DNA sequence encoding ThiI WT
SEQ ID NO: 25: Protein sequence of T5LacO repressed promoter
SEQ ID NO: 26: DNA sequence of Promoter ApFAB346
SEQ ID NO: 27: Protein sequence of ThiO
SEQ ID NO: 28: DNA sequence encoding ThiO
SEQ ID NO: 29: Protein sequence of ThiO
SEQ ID NO: 30: DNA sequence encoding ThiO
SEQ ID NO: 31: Protein sequence of IscR
SEQ ID NO: 32: DNA sequence encoding IscR
SEQ ID NO: 33: Protein sequence of ThiI truncated version
SEQ ID NO: 34: DNA sequence encoding ThiI truncated version
SEQ ID NO: 35: Protein sequence of ThiI
SEQ ID NO: 36: DNA sequence encoding ThiI
SEQ ID NO: 37: Protein sequence of ThiI
SEQ ID NO: 38: DNA sequence encoding ThiI
SEQ ID NO: 39: Protein sequence of ThiI
SEQ ID NO: 40: DNA sequence encoding ThiI
SEQ ID NO: 41: Protein sequence of ThiI
SEQ ID NO: 42: DNA sequence encoding ThiI
SEQ ID NO: 43: DNA sequence encoding apFAB71
SEQ ID NO: 44: DNA sequence encoding apFAB70
SEQ ID NO: 45: DNA sequence encoding IPTG inducible promoter
SEQ ID NO: 46: DNA sequence encoding artificial RBS sequence
SEQ ID NO: 47: DNA sequence encoding apFAB46 promoter
SEQ ID NO: 48: DNA sequence encoding apFAB306 promoter

### Working Examples

### Strains and Plasmids

Strains of Escherichia coli used herein were the following:

| | | | E. coli ThiH or | |
|---|---|---|---|---|
| | | | heterologous | E. coli ThiI WT or |
| Strain name | Plasmid | Relevant description | ThiO | truncated form |
| BS00734 | — | ΔthiP derivative of BS010131 | — | — |
| BS01013 | — | E. coli K-12 BW25113¹ parent strain: rrnB3 | — | — |
| | | ΔlacZ4787 hsdR514 Δ(araBAD)567 Δ(rhaBAD)568 | | |
| | | rph-1 | | |
| BS04565 | — | BS00734 derivative with chromosomally | — | — |
| | | integrated phosphatase from Arabidopsis thaliana | | |
| | | At5g32470² | | |
| BS04701 | — | BS04565 derivative comprising a H107Y mutation | — | — |
| | | in iscR³ | | |
| BS04782 | — | Derivative of BS04701 with complete deletion of | — | — |
| | | CyaA (recombineering). | | |
| BS05801 | — | Derivative of BS04782 with chromosomally | — | — |
| | | integrated ThiE, ThiM and ThiD genes | | |
| | | (clonetegration).⁴ | | |
| BS05850 | pBS2819 | Derivative of BS05801 comprising a plasmid with | E. coli ThiH | — |
| | | constitutive expression of ThiF, ThiS, ThiG and ThiH | | |
| | | genes from E. coli | | |
| BS06232 | pBS2819 | Derivative of BS05801 comprising a plasmid with | E. coli ThiH | E. coli ThiI WT |
| | pBS2896 | constitutive expression of ThiF, ThiS, ThiG and ThiH | | |
| | | genes from E. coli and a second plasmid with IPTG | | |
| | | inducible ThiI from E. coli | | |
| BS06278 | pBS2919 | Derivative of BS05801 comprising a plasmid with | P. putida | — |
| | | constitutive expression of E. coli genes ThiF, ThiS, | ThiO | |
| | | ThiG and ThiO heterologous gene from P. putida | | |
| BS06285 | pBS2919 | Derivative of BS05801 comprising a plasmid with | P. putida | E. coli ThiI WT |
| | pBS2896 | constitutive expression of E. coli genes ThiF, ThiS, | ThiO | |
| | | ThiG and ThiO heterologous gene from P. putida | | |
| | | and a second plasmid with IPTG inducible ThiI from | | |
| | | E. coli | | |
| BS06280 | pBS2921 | Derivative of BS05801 comprising a plasmid with | B. subtilis | — |
| | | constitutive expression of E. coli genes ThiF, ThiS, | ThiO | |
| | | ThiG and ThiO heterologous gene from B. subtilis | | |
| BS06287 | pBS2921 | Derivative of BS05801 comprising a plasmid with | B. subtilis | E. coli ThiI WT |
| | pBS2896 | constitutive expression of E. coli genes ThiF, ThiS, | ThiO | |
| | | ThiG and ThiO heterologous gene from B. subtilis | | |
| | | and a second plasmid with IPTG inducible ThiI from | | |
| | | E. coli | | |
| BS07082 | pBS3120 | Derivative of BS05801 comprising a plasmid with | P. putida | E. coli ThiI WT |
| | | constitutive expression of E. coli genes ThiF, ThiS, | ThiO | |
| | | ThiG ThiI and ThiO heterologous gene from | | |
| | | P. putida. The gene of ThiI is the WT version. | | |
| BS07083 | pBS3121 | Derivative of BS05801 comprising a plasmid with | P. putida | E. coli ThiI |
| | | constitutive expression of E. coli genes ThiF, ThiS, | ThiO | truncated |
| | | ThiG ThiI and ThiO heterologous gene from | | version (98 |
| | | P. putida. The gene of ThiI is a truncated version | | residues) |
| | | (residues 385-482). | | |
| BS06375 | pBS2967 | Derivative of BS05801 comprising a plasmid with | P. putida | E. coli ThiI WT |
| | | constitutive expression of E. coli genes ThiF, ThiS, | ThiO | |
| | | ThiG ThiI and ThiO heterologous gene from | | |
| | | P. putida. | | |
| BS06468 | pBS3001 | Derivative of BS05801 comprising a plasmid with | P. putida | E. coli ThiI WT |
| | | constitutive expression of E. coli genes ThiC, ThiE, | ThiO | |
| | | ThiF, ThiS, ThiG ThiI and ThiO heterologous gene | | |
| | | from P. putida. | | |
| BS05897 | pBS140 | Derivative of BS05801 comprising a plasmid with | E. coli ThiH | — |
| | | constitutive expression of ThiC, ThiE, ThiF, ThiS, | | |
| | | ThiG and ThiH genes from E. coli. | | |
| BS06245 | pBS140 | Derivative of BS05801 comprising a plasmid with | E. coli ThiH | E. coli ThiI WT |
| | pBS2896 | constitutive expression of ThiC, ThiE, ThiF, ThiS, | | |
| | | ThiG and ThiH genes from E. coli and a second | | |
| | | plasmid with IPTG inducible ThiI from E. coli. | | |
| BS06581 | pBS3001 | Derivative of BS05801 comprising a plasmid with | P. putida | E. coli ThiI WT |
| | pBS3038 | constitutive expression of E. coli genes ThiC, ThiE, | ThiO | |
| | | ThiF, ThiS, ThiG ThiI and ThiO heterologous gene | | |
| | | from P. putida and a second plasmid with | | |
| | | constitutive expression of ThiE and ThiD. | | |

¹Commercially available strain, e.g., from the Coli Genetic Stock Center; http://cgsc2.biology.yale.edu/KeioList.php
²WO2017103221A1
³WO2019012058A1
⁴Method is described elsewhere: https://pubmed.ncbi.nlm.nih.gov/24050148/

Plasmids used herein were as follows:

| Plasmid ID | IPTG inducible expression of | Origin | Antibiotic marker | DNA sequence ID |
|---|---|---|---|---|
| pBS2896 | ThiI [SEQ. ID No. 24] | SC101 | Tet^{R} | [SEQ. ID No. 24 and 25] |
| | overexpression plasmid from | | | |
| | a T5LacO repressed promoter | | | |
| | [SEQ ID No. 25]. | | | |

| Plasmid ID | Constitutive expression of | Origin | Antibiotic marker | DNA sequence ID |
|---|---|---|---|---|
| pBS2819 | Thiamine pathway genes | p15A | Spec^{R} | [SEQ. ID No. 6, 8, |
| | from E. coli ThiF, ThiS, ThiG, | | | 12, 10 and 43] |
| | ThiH [SEQ. ID No. 6, 8, 12, 10, | | | |
| | respectively] under | | | |
| | constitutive apFAB71 | | | |
| | promoter (SEQ. ID No. 43) | | | |
| pBS2919 | Thiamine pathway genes | p15A | Spec^{R} | [SEQ. ID No. 6, 8, 12, 30 |
| | from E. coli ThiF, ThiS, ThiG | | | and 43] |
| | and ThiO from P. putida in | | | |
| | native form [SEQ. ID No. 6, 8, | | | |
| | 12, 30, respectively] under | | | |
| | constitutive apFAB71 | | | |
| | promoter (SEQ. ID No. 43) | | | |
| pBS2921 | Thiamine pathway genes | p15A | Spec^{R} | [SEQ. ID No: 6, 8, 12, 28 |
| | from E. coli ThiF, ThiS, ThiG | | | and 43] |
| | and ThiO from B. subtilis in | | | |
| | native form [SEQ. ID No: 6, 8, | | | |
| | 12, 28, respectively] under | | | |
| | constitutive apFAB71 | | | |
| | promoter (SEQ. ID No. 43) | | | |
| pBS3120 | Thiamine pathway genes | p15A | Spec^{R} | [SEQ. ID No: 6, 8, 12, |
| | from E. coli ThiF, ThiS, ThiG, | | | 24, 30, 43] |
| | ThiI WT and ThiO from | | | |
| | P. putida in native form [SEQ. ID | | | |
| | No: 6, 8, 12, 24, 30, | | | |
| | respectively] under | | | |
| | constitutive apFAB71 | | | |
| | promoter (SEQ. ID No. 43) | | | |
| pBS3121 | Thiamine pathway genes | p15A | Spec^{R} | [SEQ. ID No: 6, 8, 12, |
| | from E. coli ThiF, ThiS, ThiG, | | | 34, 30, and 43] |
| | ThiI truncated version and | | | |
| | ThiO from P. putida in native | | | |
| | form [SEQ. ID No: 6, 8, 12, 34, | | | |
| | 30, respectively] under | | | |
| | constitutive apFAB71 | | | |
| | promoter (SEQ. ID No. 43). | | | |
| pBS2967 | Thiamine pathway genes | p15A | Spec^{R} | [SEQ ID No: 6, 8, 12, 24, |
| | from E. coli ThiF, ThiS, ThiG, | | | 29 and 43] |
| | ThiI WT and ThiO from P. | | | |
| | putida in native form [SEQ. ID | | | |
| | No: 29] under constitutive | | | |
| | apFAB71 promoter (SEQ. ID | | | |
| | No. 43) | | | |
| pBS3001 | Thiamine pathway genes | p15A | Spec^{R} | [SEQ. ID No: 6, 8, 12, |
| | from E. coli ThiF, ThiS, ThiG, | | | 24, 29, 2, 22 and 43] |
| | ThiI WT, ThiO from P. putida | | | |
| | in native form, ThiC and ThiE | | | |
| | [SEQ. ID No: 22] under | | | |
| | constitutive apFAB71 | | | |
| | promoter (SEQ. ID No. 43) | | | |
| pBS140 | Thiamine pathway genes | pBR322 | Kan^{R} | [SEQ. ID No: 2, 22, 6, 8, |
| | from E. coli ThiC, ThiE, ThiF, | | | 12, 10, 47, 14 and 43] |
| | ThiS, ThiG, ThiH [SEQ. ID No: | | | |
| | 10] under constitutive | | | |
| | apFAB46 promoter (SEQ ID | | | |
| | No: 47) as well as ThiM and | | | |
| | ThiC under constitutive | | | |
| | apFAB71 promoter (SEQ ID | | | |
| | No: 43) | | | |
| pBS3038 | Thiamine pathway genes | pSC101 | TetR | [SEQ. ID No: 6, 8, 12, |
| | from E. coli ThiE, ThiD [SEQ ID | | | 10, 48] |
| | No: 6, 8, 12, 10, respectively] | | | |
| | under constitutive apFAB306 | | | |
| | promoter (SEQ ID No. 48) | | | |

### Analytical Procedures

### Procedure I: Optical Densities Measurements

To measure optical densities (OD) of a cell culture as cuvette OD at 600 nm (cOD₆₀₀), the culture was diluted 10-fold with dH₂O to a final volume of 1 ml and transferred to a 1.5 ml transparent cuvette with 10 mm pathlength. The diluted culture was measured at 600 nm and 10 mm pathlength on a mySPEC (VWR). If the diluted culture was measured to cOD600>0.4, the culture was further diluted 10-fold and remeasured.

### Procedure II: Thiochrome Assay for Thiamine Quantification

For quantification of thiamine in supernatant samples from small-scale cultivations, the thiochrome assay was used following the assay described in section III of the methods in WO2017103221A1. Specifically, the supernatant from each culture was diluted alongside >20 thiamine standards in the concentration range of 0 μM (mg/L) to 60 μM (15.9 mg thiamine/L) prepared in Milli-Qwater. 50 μL of supernatant and each of the thiamine standards were then added to a well of a 96-well microtiter plate. To each well, 100 μl of 4 M potassium acetate are added. Samples are then oxidized by the addition of 50 pl freshly prepared 3.8 mM potassium ferricyanide in 7 M NaOH. The solutions are mixed by pipetting and quenched by addition of 50 pl fresh 0.06% H₂O₂ in saturated KH₂PO₄. Samples are neutralized with 6M HCl and fluorescence is measured at 444 nm after excitation at 365 nm. The thiamine concentrations per well/sample are estimated based on comparison to standard curves included in the derivatization plate.

### Procedure III: LC-MS Method for Quantification of Thiamine and Precursors

### Chemicals and Reagents

Commercial standards for the analytes of interest were purchased from suppliers listed in Table 1 below. Water (H₂O) was purchased from Honeywell and methanol (MeOH), ascorbic acid and ammonium bicarbonate (NH4HCO3) from Sigma-Aldrich. Ammonium hydroxide was purchased from Carl Roth. Stock solutions of the analytes and internal standard were prepared in H₂O:MeOH (50:50, v/v)+0.1% ascorbic acid to a concentration of 1 mg mL-1. Working standard solutions of the stock solutions were then prepared in H₂O:MeOH (50:50, v/v)+0.1% ascorbic acid. Calibration curves in the concentrations of 0.1, 0.25, 0.5, 1, 2.5, 5, 10, 25, 50, 100, 250, 500 and 1000 ng mL-1 were prepared in H₂O:MeOH (50:50, v/v)+0.1% ascorbic acid and the internal standard 13C4-THI was added to the each calibration curve sample to correspond to a final concentration of 0.5 ng mL-1. A working standard solution of the internal standards was prepared in H₂O:MeOH (50:50, v/v)+0.1% ascorbic acid to contain 50 ng mL-1 of 13C4-THI. This internal standard solution (ISTD MIX) was added to each sample for quality control and normalization purposes.

### Sample Preparation

Before analyses, the samples from the bioreactors are diluted and a mixture of internal standards (ISTD MIX) is added to correct for possible technical variation. To be able to quantify the analytes as accurately as possible, two different dilution factors are applied. For analytes present in lower concentrations, a dilution factor of 1:50 is applied, while the analytes present in higher concentrations are quantified using a dilution factor of 1:1500. The 1:1500 dilution is prepared in two steps. First a 1:15 dilution is achieved by pipetting 980 L of H₂O:MeOH (50:50, v/v)+0.1% ascorbic acid into an Eppendorf tube and by adding 70 ?L of the supernatant. The tube is then vortex mixed thoroughly. The final 1:1500 dilution is achieved by pipetting 980 μL of H₂O:MeOH (50:50, v/v)+0.1% ascorbic acid into a glass vial and by adding 10 ?L of the 1:15 diluted sample as well as 10 μL of the ISTD MIX. The solution is again vortex mixed. The 1:50 dilution is achieved by pipetting 970 μL of H₂O:MeOH (50:50, v/v)+0.1% ascorbic acid into a glass vial and by adding 20 μL of the original sample as well as 10 ML of the ISTD MIX. The solution is again vortex mixed.

### Liquid Chromatography-Tandem Mass Spectrometry

The samples are randomized after sample preparation and analysed by ultra-high performance liquid chromatography (Infinity II, Agilent Technologies) coupled to tandem mass spectrometry (6470 Triple Quadrupole, Agilent Technologies) using electrospray ionization in positive ion mode. Selected reaction monitoring is used for quantifying the analytes and fragmentor voltages, collision energies and cell accelerator voltages were optimized for each ion transition.

The analytes are separated chromatographically before they enter the mass spectrometer. This is done using a XBridge Premier BEH C18 VanGuard FIT Column (4.6 mm×100 mm, particle size 2.5 Mm, Waters Corporation) and H₂O+10 mM NH4HCO3 (pH 8.8, adjusted with NH40H) as eluent A and MeOH+10 mM NH4HCO3 (pH 8.8, adjusted with NH₄OH) as eluent B with a flow rate of 0.6 mL min-1. The elution gradient is as follows: 0-2.6 min 0% B to 20% B, 2.6-3 min 20% to 30% B, 3-3.5 min 30% B to 95% B, 3.5-5.5 min 95% B, 5.5-5.6 min 95% B to 0% B. After each run, the column is re-equilibrated at 0% B for 2.4 min. The injection volume for each sample is 2 μL. All data is acquired using the MassHunter Acquisition software (Version 10.0, Build 10.0.142) by Agilent Technologies and the retention times for each of the analytes are shown in Table 1.

TABLE 1
Abbreviations and suppliers of commercial standards including retention times.

| Analyte | Abbreviation | Supplier | Rₜ (min) |
|---|---|---|---|
| (4-amino-2-methylpyrimidin-5- | HMP-PP | Toronto Research | 1.8 |
| yl)methyl Trihydrogen Diphosphate | | Chemicals | |
| (4-amino-2-methylpyrimidin-5- | HMP-P | Toronto Research | 2.2 |
| yl)methyl Dihydrogen Phosphate | | Chemicals | |
| Phosphorylated sulfurol or thiazole | THZ-P | In-house synthesis | 3.1 |
| Thiamine pyrophosphate | TPP | Sigma Aldrich | 3.5 |
| Thiamine monophosphate | TMP | Sigma Aldrich | 3.9 |
| Thiamine hydrochloride | THI | Sigma Aldrich | 4.5 |
| ¹³C₄-thiamine hydrocholoride | ¹³C₄-THI | Sigma Aldrich | 4.5 |
| 5-(2-hydroxyethyl)-4-methyl-1,3- | cTHZ | Finetech | 4.7 |
| thiazole-2-carboxylic acid | | | |
| (4-amino-2-methyl-5- | HMP | Fluorochem | 5.1 |
| pyrimidinyl)methanol | | | |
| Sulfurol or thiazole | THZ | Sigma Aldrich | 5.9 |

### Data Processing

All data is processed using the MassHunter Quantitative Analysis software (Version B.09.00, Build 9.0.647.0) by Agilent Technologies. The peak areas for the analytes of interest are normalized against the peak areas of the internal standard ¹³C4-THI. Data quality is ensured by evaluating technical replicates of a specific fermentation sample as well as commercial standards. The quantified concentration of THI in these samples is required to remain within ±10% for the data set to be approved.

### Molecular Biology Techniques

Standard techniques were used for DNA isolation, amplification, purification and cloning (restriction digestion, ligation), transformation and the like. Such techniques are well known in the art and standard protocols can be found in: Maniatis et al., 1982 Molecular Cloning, Cold Spring Harbor Laboratory, Plainview NY. And M. Green, J. Sambrook (2012) Molecular Cloning: a laboratory manual. 4th Edition, Cold Spring Harbor Laboratory Press, CSH, NY., which are both hereby incorporated by reference in their entireties.

### Example 1—Construction of Strain Carrying IscR and CyaA Mutations

Using the ΔThiP mutant BS00734 as starting strain, a PCR cassette of the Arabidopsis thaliana phosphatase was introduced into the chromosome at location KO-176 via the “clonetegration” method as described in PCT/EP2022/069711. The resulting strain BS04565 was used as the starting point for mutagenesis of IscR. This transcription factor regulates the expression of dozens of genes involved in the biosynthesis of FeS clusters. By mutating IscR to favour the apoprotein formation, we aim to improve thiamine production. Accordingly, iscR was mutated in BS04565 by multiplex automated genome engineering (MAGE) as described in Methods in Enzymology, 498, 409-426, 2011 using the DNA oligo shown in Table 3, yielding strain BS04701.

CRP is activated as a transcriptional regulator by binding to its allosteric activator cyclic AMP (cAMP) which is produced by the enzyme adenylate cyclase (encoded by cyaA). Under low-glucose conditions adenylate cyclase is activated by the phosphorylated state of glucose-specific enzyme IIA or EIIA (encoded by crr). To effectively eliminate cAMP, the gene cyaA was completely removed from the chromosome of strain BS04701 using methods known in the art and replaced with only a short FRT sequence. One method to achieve this removal of the cyaA gene is by generating a DNA fragment carrying a Kanamycin resistance gene flanked by homologous regions of cyaA and transforming strain BS04701 (carrying the λRed recombinase genes expressed from an inducible promoter) with this DNA cassette. One protocol for such a method can be found in Datsenko, K. A. and Wanner, B. L., One-step inactivation of chromosomal genes in Escherichia coli K-12 using PCR products, PNAS, 2000, 97(12):6640-5, DOI:10.1073/pnas.120163297.

TABLE 3
Oligos used to introduce and check mutations

| Mutation | Oligo | | Resulting |
|---|---|---|---|
| description | name | Oligo sequence | strain |
| IscR | moBS151 | TCGCCTAAAGTAATGTTGTTGAGAAAAC | BS04701 |
| mutation | (MAGE) | CGGTGAGACGGTCGCTCAAATCACGCCA | |
| H107Y | | CAGCGCGTaGGTCAGGCATTTATCGCCG | |
| | | CCCTGG | |
| IsCR FW | OBS3225 | AATGCTTGACGTTGCGCTCAA | — |
| | (Check) | | |
| IsCR RV | OBS3226 | GTGGTTGCGGAGTAGTCGAGAT | — |
| | (Check) | | |
| CyaA | OBS4519 | GTTGGCGGAATCACAGTCATGACGGGT | BS04782 |
| deletion | (KO) | AGCAAATCAGGCGATACGTCTTGGCAT | |
| FW | | TACACGTCTTGAGCGATTGTG | |
| CyaA | OBS4520 | CGGATAAGCCTCGCTTTCCGGCACGTT | BS04782 |
| deletion | (KO) | CATCACGAAAAATATTGCTGTAATGGG | |
| RV | | AATTAGCCATGGTCCATATGAAT | |
| CyaA FW | OBS4046 | CGAAATATCCCGAATGCCGCGT | — |
| | (Check) | | |
| CyaA RV | OBS4047 | CGTCGCACGGCACCATATTCCA | — |
| | (Check) | | |

Successful introduction of the desired mutations was verified by PCR amplification of the region followed by Sanger sequencing using the oligos in Table 3.

### Example 2—Construction of Strains Carrying Genes for Providing Thiamine Production

ThiEMD were introduced into the chromosome using the clonetegration method as described in PCT/EP2022/069711. The promoter of ThiEMD is apFAB70 (SEQ ID No: 44), and each gene has a specific RBS sequence (SEQ ID NO: 46).

pBS2896: An IPTG-inducible (Promoter SEQ ID NO: 45) transgene encoding ThiI WT (SEQ ID NO: 24) was cloned on plasmid pBS2896 by amplification of the gene from E. coli's chromosome and a ribosome binding site using Phusion U polymerase (Thermo Fischer Scientific) following manufacturer's protocol and using primers containing Uracils for recognition by USER restriction enzymes. Similarly, a plasmid backbone carrying origin pSC101 and a tetracycline resistance cassette as well as the promoter sequence was amplified. DNA fragments were digested and ligated using USER enzyme (New England Biolabs) and T4 ligase (Thermo Fischer Scientific) following the manufacturer's protocols. These mixtures were introduced by electroporation into BS05801 and transformed cells were grown on selective LB agar supplemented with tetracycline overnight at 37 degrees Celsius.

pBS2819: A plasmid encoding genes thiF, thiS, thiG and thiH (SEQ ID Nos. 6, 8, 12, 10, respectively) driven by constitutive promoter apFAB71 (SEQ ID No: 43) was cloned on a plasmid backbone carrying a spectinomycin resistance cassette and p15A origin of replication using USER cloning methods as described above for pBS2896 construction. Each gene was proceeded by a synthetic ribosome binding site (RBS) sequence.

pBS2919: A plasmid encoding genes thiF, thiS, thiG and ThiO from P. putida (SEQ ID Nos.6, 8, 12, 30, respectively) driven by constitutive promoter apFAB71 (SEQ ID No: 43) was cloned on a plasmid backbone carrying a spectinomycin resistance cassette and p15A origin of replication using USER cloning methods as described above for pBS2896 construction. Each gene was proceeded by a synthetic RBS sequence.

pBS2921: A plasmid encoding genes thiF, thiS, thiG and ThiO from B. subtilis (SEQ ID Nos. 6, 8, 12, 28, respectively) driven by constitutive promoter apFAB71 (SEQ ID No: 43) was cloned on a plasmid backbone carrying a spectinomycin resistance cassette and p15A origin of replication using USER cloning methods as described above for pBS2896 construction. Each gene was proceeded by a synthetic RBS sequence.

pBS3120: A plasmid encoding genes thiF, thiS, thiG, ThiI (WT form of 1,449 bp) and ThiO from P. putida (SEQ ID Nos. 6, 8, 12, 24, 30, respectively) driven by constitutive promoter apFAB71 (SEQ ID No: 43) was cloned on a plasmid backbone carrying a spectinomycin resistance cassette and p15A origin of replication using USER cloning methods as described above for pBS2896 construction. Each gene was proceeded by a synthetic RBS sequence.

pBS3121: A plasmid encoding genes thiF, thiS, thiG, ThiI (truncated form of 300 bp) and ThiO from P. putida (SEQ ID Nos. 6, 8, 12, 34 and 36 enzyme, respectively) driven by constitutive promoter apFAB71 (SEQ ID No: 43) was cloned on a plasmid backbone carrying a spectinomycin resistance cassette and p15A origin of replication using USER cloning methods as described above for pBS2896 construction. Each gene was proceeded by a synthetic RBS sequence.

Transformation: Plasmids pBS2819 (alone or with pBS2896), pBS2919 (alone or with pBS2896), pBS2921 (alone or with pBS2896), pBS3120 or pBS3121, were introduced into background strain BS05801 via electroporation transformation protocols that are well known in the art and transformant strains were selected on selective agar plates containing spectinomycin (pBS2819, pBS2919, pBS2921, pBS3120 or pBS3121) and/or tetracycline (pBS2896).

### Example 3: Replacing ThiH with ThiO and Balancing Expression of ThiI for Increase in Thiamine Titer Compared to ThiH or ThiO Alone

BS05801 strain can produce thiamine upon feeding both precursors HMP and THZ because ThiD converts HMP to HMP-PP, whereas ThiM converts THZ to cTHZ-P. Both precursors HMP-PP and cTHZ-P are ligated by ThiE to produce thiamine monophosphate (TMP) and thiamine (a single copy phosphatase gene in the chromosome converts >98% TMP to thiamine). In standard small-scale cultivations (see below), addition of 500 μM HMP and 500 μM THZ to BS05801 results in a titer of ˜20 mg/L thiamine.

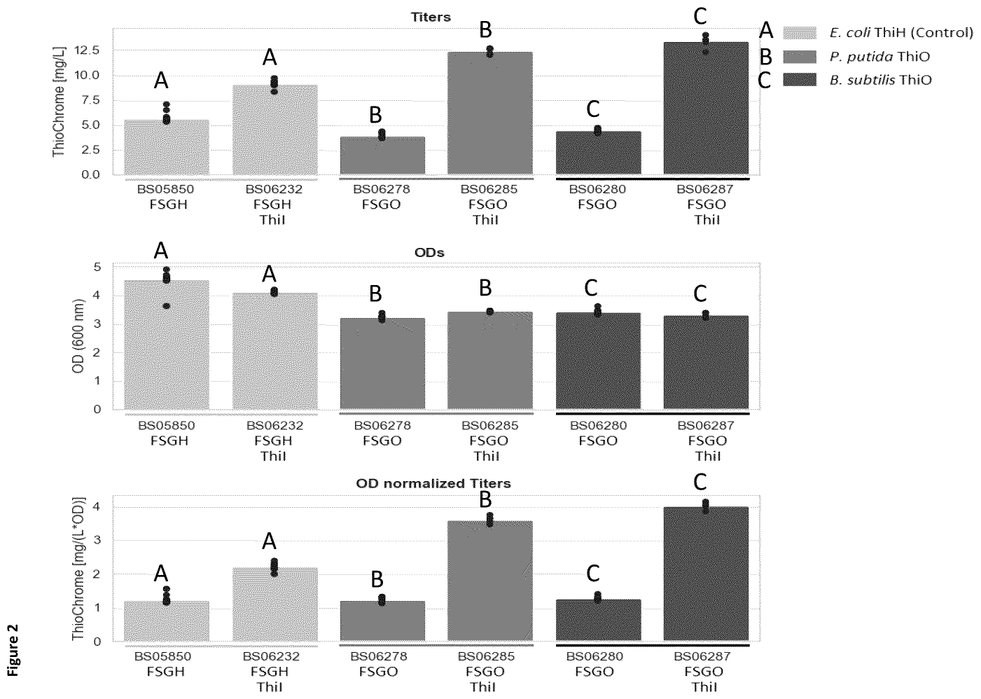

When feeding strain BS05801 with HMP alone while co-expressing genes involved in THZ formation (ThiF, ThiS, ThiG and ThiH), it is possible to probe the capacity of THZ production based on thiamine quantification. Prior work in our laboratory established that, to maximize THZ production, it is crucial not only to over-express ThiF, ThiS, ThiG and ThiH, but also to balance their expression carefully using specific RBS sequences such as a sequence comprising SEQ ID NO: 46 (data not shown). Our best strain over-expressing these 4 genes (BS05850) produces around 6 mg/L thiamine from 2 g/L glucose and 500 μM HMP (FIG. 2 top panel). Yet ThiI co-expression (BS06232) increases THZ production by 30% to 9 mg/L thiamine (FIG. 2 top panel). Interestingly, although ThiH can be replaced by ThiO from different organisms (BS06278 or BS06280) to produce ˜4.5 mg/L thiamine, ThiI co-expression increases titer by >300% to ˜13 mg/L in both cases (BS06285 and BS06287). Such improvements are >30% higher than the combination of native ThiH and ThiI (˜9 mg/L). The final OD values for the 4 strains containing ThiO are very similar, but ˜30% lower than that of the thiH controls−/+ThiI (FIG. 2 middle panel), suggesting that the production per biomass is even higher for the ThiO strains. In fact, the four ThiO strains have 75 and 100% higher productivity per cell, as shown for OD-normalized titers (FIG. 2 bottom panel). All strains were cultivated in 400 μL of mMOPS fed with 500 μM HMP without or with IPTG. ThiI expression was IPTG inducible and the IPTG concentration that gives maximal activity was chosen for each strain, whereas ThiFSGH or ThiFSGO expression was constitutive. The strains were grown in deep well plates for 24 hours at 37 degrees C. shaken at 275 rpm, after which thiamine production was evaluated using a the thiochrome assay. The resulting thiamine titers are shown in FIG. 2. Bars illustrate the median thiamine production value (height), black dots show thiamine production from individual replicate cultures. A total of 4 replicates were used per strain.

### Example 4: Using Rhodanese Domain of ThiI for Producing Thiamine

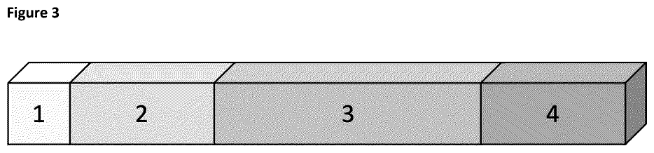

ThiI is a versatile gene that is not only involved in thiamine biosynthesis (transfer of sulfur from IscS to the sulfur carrier protein ThiS, forming ThiS-thiocarboxylate), but it also catalyzes the ATP-dependent transfer of sulfur to tRNA as part of a modification (4-thiouridine) that works as a near-UV photosensor in E. coli. ThiI is a very long protein that in E. coli is composed of 482 amino acid residues, which form different functional domains. FIG. 3 shows the four domains that are present in ThiI from E. coli and other organisms. The first three domains are involved in binding to ferredoxin, ATP and tRNA, whereas the fourth domain is proposed to exclusively work for thiazole biosynthesis.

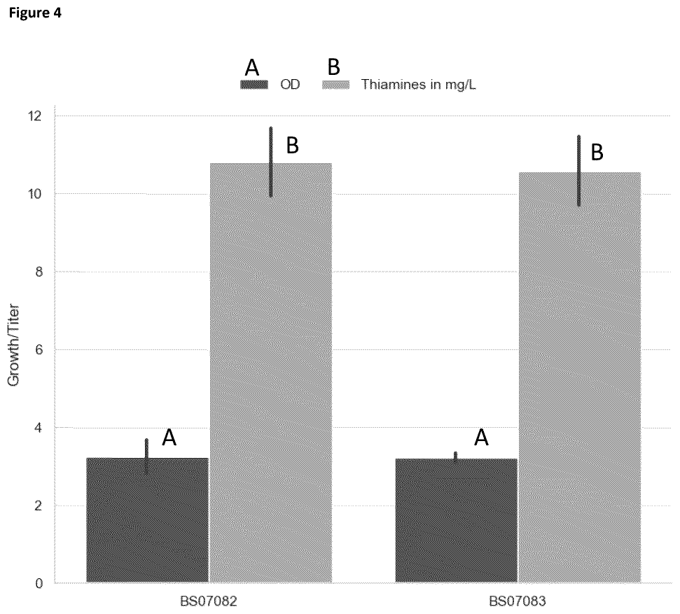

To show that ThiI WT is not needed to make THZ (as measured by thiamine thanks to the co-expression of ThiD and ThiE on the chromosome), ThiI was truncated at the N-terminus to remove the first three domains, leaving a truncated version of 98 amino acids (residue 1384 was changed to methionine, leaving the remaining C-terminus intact until the last amino acid P482). ThiI WT or truncated versions were then introduced into an operon containing already the E. coli genes ThiF, ThiS, ThiG and P. putida ThiO with defined RBS sequences, resulting in an operon of five genes that are constitutively expressed and require no addition of IPTG. The RBS sequence of ThiI was also optimized in both cases, resulting in the ThiI WT (BS07082) or truncated (BS07083) versions. Both strains were cultivated in 400 μL of mMOPS fed with HMP in deep well plates for 24 hours at 37 degrees C. shaken at 275 rpm, after which thiamine production was evaluated using a the thiochrome assay. The resulting thiamine titers of ˜11 mg/L from 2 g/L glucose and 500 μM HMP are shown in FIG. 4. Bars illustrate the median thiamine titer and final OD values (height), indicating that both strains have very similar production and growth patterns. A total of 4 replicates were used per strain. Thus, the rhodanese domain of E. coli ThiI and likely of other organisms is sufficient to produce thiamine.

### Example 5: Bench-Top Fed-Batch Fermentation of Strains Over-Expressing ThiI and ThiO

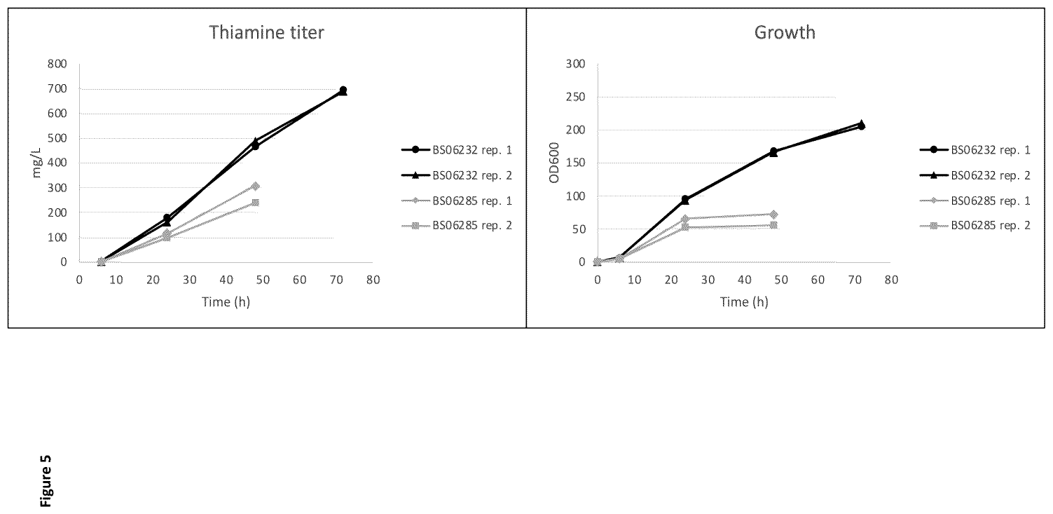

Small scale, over-expression of ThiI with two heterologous ThiOs showed a ˜30% greater thiamine titer than with native ThiH, but a 20% decrease in growth (FIG. 2). This effect is independent of thiI because the ThiFSGO strains without thiI exhibited an even lower final OD of 30% compared to reference strain ThiFSGH. This data suggests that ThiO over-expression causes growth defects. In contrast to the small-scale data, one of the strains with the best ThiO+thiI combination (BS06285) performed worse than the reference strain (BS06232) in 0.5 L fed-batch fermentations (FIG. 5). Growth of BS06285 stopped at 48 h, reaching an average thiamine titer of ˜270 mg/L, which is 60% lower than that of BS06232 at 48 h (480 mg/L) or 300% lower at 72 h (˜700 mg/L). The fermentation data indicates that ThiO expression needs to be tuned appropriately to avoid issues.

### Example 6: Optimization of ThiO Expression Levels

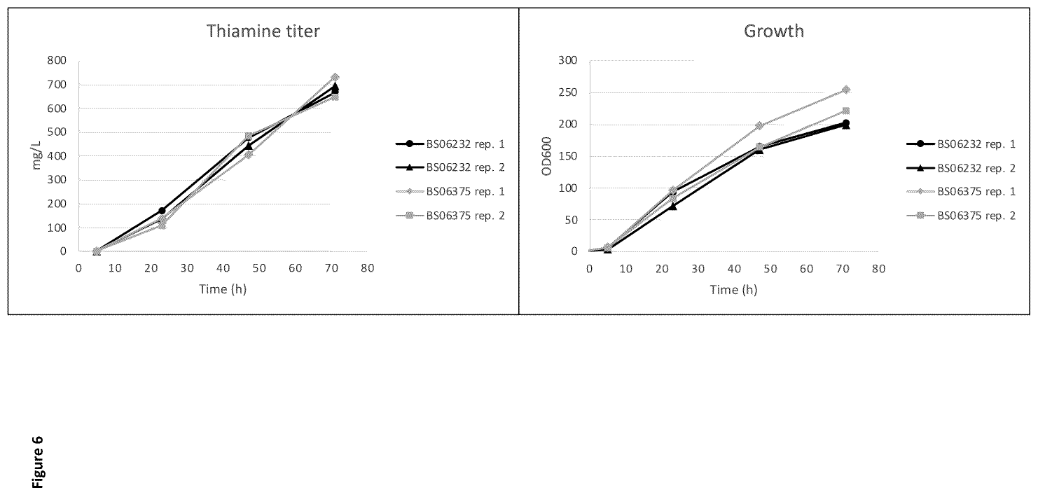

Next, the expression level of ThiO was optimised by combining thiI and ThiO in one operon to avoid having two separate plasmids for ThiFSGO and ThiI. Thus, the thiI gene was introduced between ThiG and ThiO, resulting in the synthetic thiamine operon ThiFSGIO in a single plasmid (pBS2967). The best strain with the new operon was then fermented (BS06375), resulting in an average thiamine titer of ˜650 mg/L at 72 h, which is very similar to that of reference strain BS06232 (FIG. 6). Growth of BS06375 was ˜25% higher than that of BS0232 (240 vs. 200 OD). The strain with the synthetic thiamine operon seems to have a proper co-expression of ThiO because growth and production are not affected.

### Example 7: Over-Expression of ThiI with a Native Thiamine Pathway Harbouring ThiH

Either ThiC or ThiC+ThiE were introduced into the synthetic operon ThiFSGIO. The starting ATG sequence of all five downstream genes of thiC overlaps with the bases at the end of the upstream gene suggesting that the co-expression of the thiamine genes is coupled and optimal for catalysis. In small scale experiments, we found that the plasmid configuration producing most thiamine de novo from glucose was ThiFSGIOCE (pBS3001).

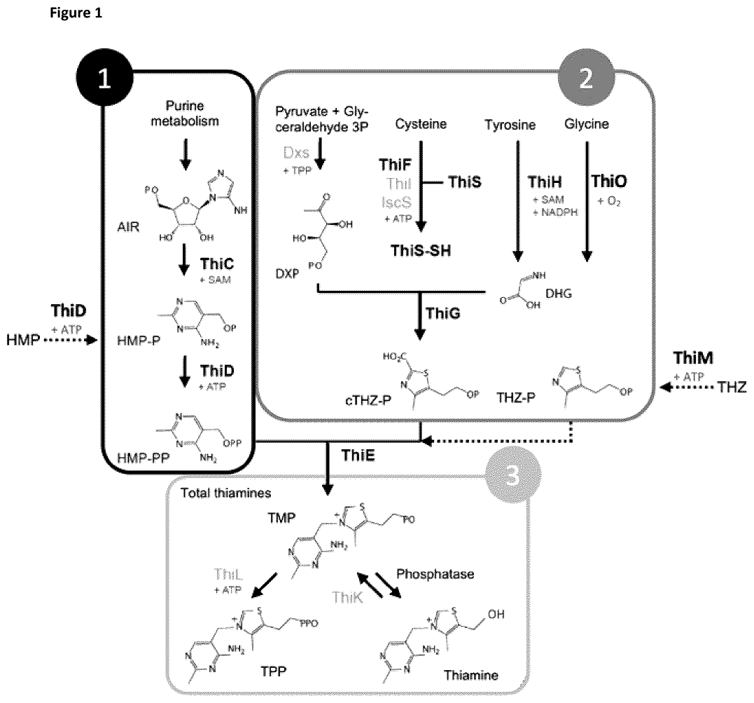

Background strain BS05801 containing a single copy of the synthetic operon ThiEMD and phosphatase on the chromosome (FIG. 1, module 3) was transformed with either the synthetic (pBS3001) or native thiamine operon plasmid (pBS140), resulting in strains BS06468 and BS05897, respectively. The latter strain was also transformed with the thiI plasmid, resulting in strain BS06245.

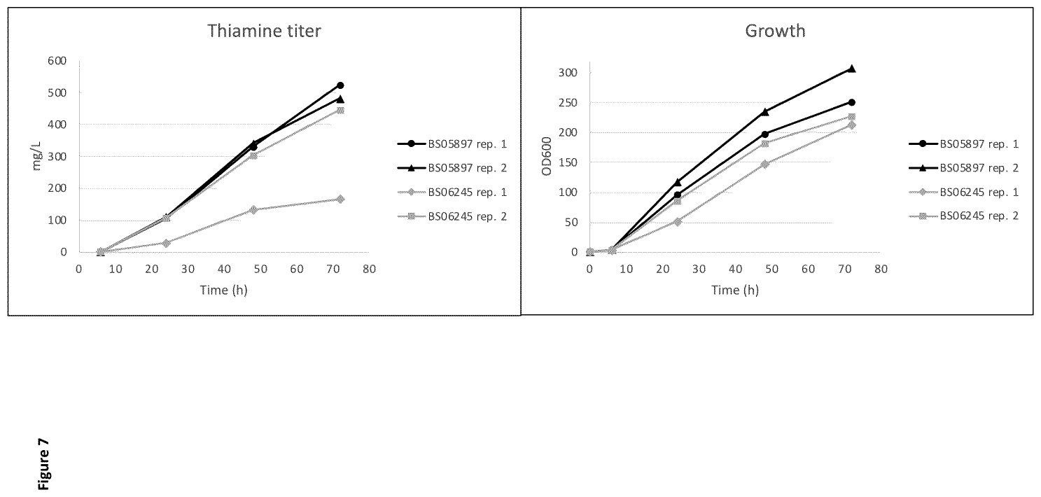

Fermented strains having the native operon without (BS05897) or with (BS06245) thiI, showed similar thiamine titers of ˜450 mg/L at 72 h (FIG. 7). However, one of the replicates of the thiI-containing strain showed poor production, indicating that thiI is not contributing to improving thiamine production. This could be because ThiC provokes the cell to have a higher demand for biosynthesizing iron-sulfur (FeS) clusters from cysteine. Likewise, in the thiazole pathway, IscS and thiI mobilize sulfur from cysteine for the thiazole moiety. In addition, the radical s-adenosyl-methionine (SAM) enzyme thiH from the native pathway also requires FeS clusters as SAM like ThiC does. Therefore, in a de novo thiamine strain, there could be a competition for sulfur, cysteine, and methionine for making both FeS clusters and SAM between Module 1 (ThiC) and Module 2 (ThiI/IscS and thiH). Consequently, replacing ThiH by ThiO leads to better thiamine titer in the fermenter.

Accordingly this example shows that over-expression of ThiI with a native thiamine pathway harbouring thiH has no positive effect on thiamine production.

### Example 8: Over-Expression of ThiI with a Synthetic Thiamine Pathway Harbouring ThiO

Since the presence of thiI in BS06245 caused poor performance in one replicate (suggesting genetic instability), while the other replicate performed like both replicates of strain BS05897 (FIG. 7), we compared the latter one (BS06245 rep 2) carrying native thiamine operons ThiCEFSGH/ThiMD free of thiI to strain BS06468 having ThiFSGIOCE as synthetic thiamine operon under the same fermentation conditions.

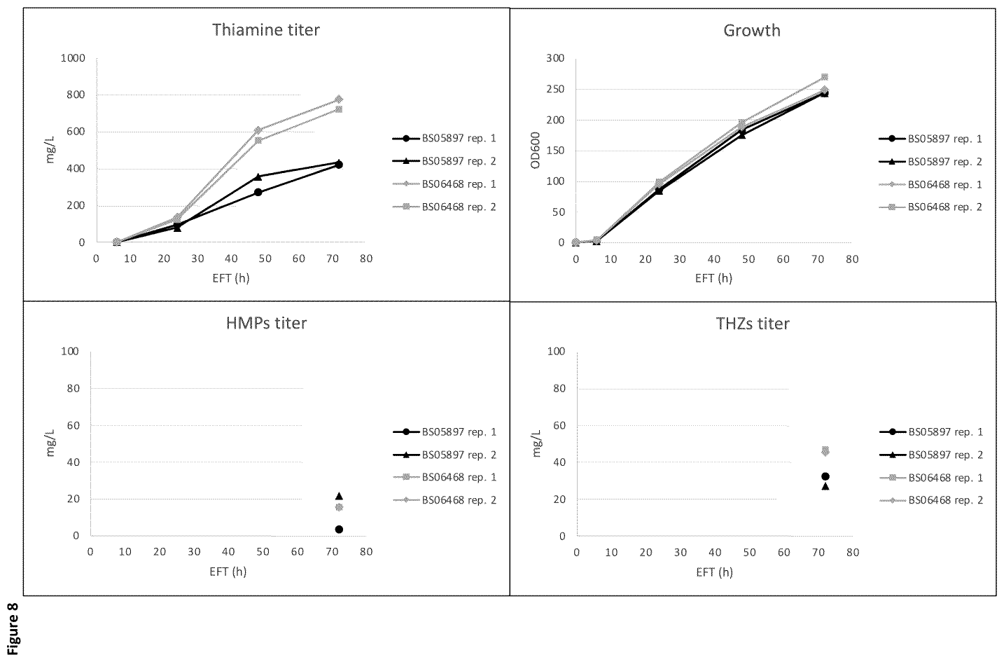

FIG. 8 shows that both replicates of BS06468 displayed a thiamine titer of ˜750 mg/L at 72 h, which is 74% higher than the reference strain BS05897 at ˜430 mg/L, with practically no differences in growth and only minor differences in the accumulation of both precursors including 4-amino-5-hydroxymethyl-2-methylpyrimidines (HMPs) and thiazoles (THZs) at ˜20 and ˜40 mg/L, respectively (FIG. 8). Thus, such improvement may be possible to reach with the synthetic ThiI+ThiO combination likely due to a less demanding and complex mechanism of ThiO compared to ThiH. Accordingly, as shown above, the expression of ThiO needs to be adapted for optimal performance.

Accordingly this example shows that over-expression of ThiI and a synthetic thiamine pathway harbouring ThiO has a significant effect on thiamine production.

### Example 9: Capacities of Thiamine Pathway Modules 1, 2 or 3

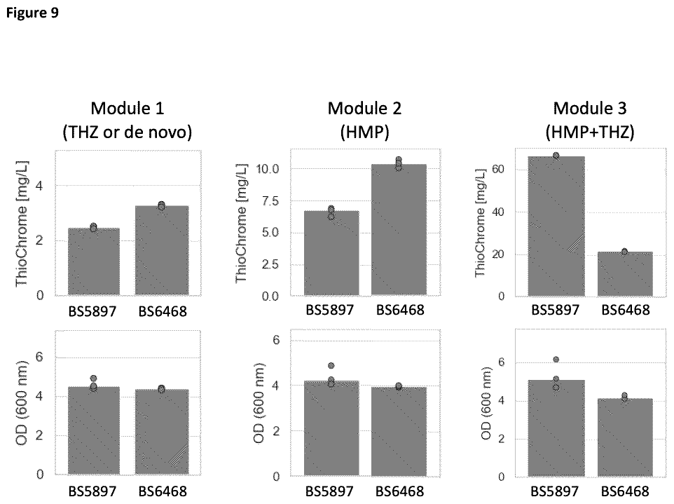

To better understand the capacity of all modules 1, 2 or 3, strains BS05897 and BS06468 were fed with THZ, HMP or HMP and THZ precursors, respectively, and quantified for thiamine production in small-scale experiments. FIG. 9 shows that, regardless of strain, the thiamine titer of Module 2 is ˜3-fold higher than Module 1. Conversely, the titer by Module 3 is 2- or 10-fold higher than that of Module 2 in BS06468 or BS05897, respectively.

Overall, the data above shows that Module 1 (ThiC and ThiD for respective formation of HMP-P and HMP-PP) is limiting, followed by Module 2 (ThiFSGH or ThiFSIGO) that ultimately produces cTHZ-P. Module 3 (ThiEMD+phosphatase) is not limiting Module 1 or 2 because kinases are very efficient enzymes, which in this case convert HMP to HMP-P and HMP-PP (ThiD) or THZ to THZ-P (ThiM). Whereas ThiE is the enzyme responsible for ligating HMP-PP and cTHZ-P or THZ-P to form thiamine monophosphate (TMP), the phosphatase converts TMP to thiamine. Thiamine diphosphate (TPP) is formed by the phosphorylation of TMP by kinase ThiL, which is present in the chromosome, but not expressed to high levels to enable efficient phosphorylation of TMP towards TPP.

In terms of strain performance, compared to BS05897, BS06468 has a ˜20% higher de novo titer (3.2 vs. 2.5 mg/L), ˜30% higher Module 2 titer (˜10 vs. 7 mg/L), but ˜3-fold lower Module 3 titer (22 vs. 66 mg/L). This large difference could be attributed to the higher over-expression of kinases ThiM and ThiD from the pBS140 plasmid in strain BS05897.

To assess the real performance of all modules in fermentation conditions, strain BS06468 was de novo cultivated without precursor addition, and with the addition of either HMP (Module 2) or HMP+THZ (Module 3). Likewise, strain BS06468 was transformed with a plasmid encoding ThiE and ThiD and fermented in the presence of HMP and THZ to determine the full potential of Module 3 in such strain.

Accordingly, this example shows that the main limiting factors for thiamine biosynthesis is ThiC (module 1), followed by thiazole formation (module 2).

### Example 10: Performance of the 3 Modules of the Best Producing Thiamine Strains

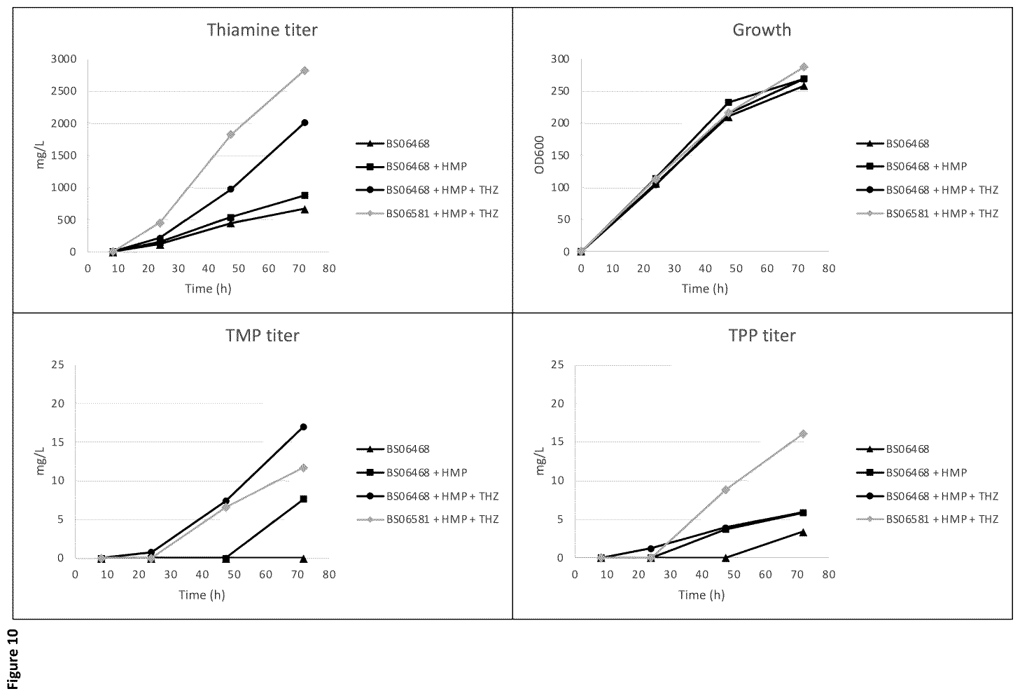

FIG. 10 shows a de novo thiamine titer of ˜700 mg/L at 72 h. This is ˜25% lower than when fed with HMP (˜900 mg/g). The addition of both HMP and THZ precursors together increased the thiamine titer to 2000 mg/L or 2 g/L, which is almost 3 and 2-fold higher than Module 1 and 2, respectively. Thus, as was observed in the small-scale feeding experiments, Module 3 is the most efficient module, followed by Module 2 and finally Module 1. Further, over-expression of ThiD and ThiE in strain BS06581 fermented in the presence of HMP and THZ reached a thiamine titer of 2.9 g/L, indicating that “Module 3” can be improved straightforwardly. Finally, we observed very low accumulation of TMP and TTP in all cases. For example, 12-17 mg/L of TMP and 6-16 mg/L of TTP were detected in supernatants of the strains fed with both precursors at 72 h (Module 3), indicating that the phosphatase expression is optimal even under high amounts of precursors. In other words, the microbial cell factories produce ˜99% thiamine and ˜0.1% of thiamine in either phosphorylated form.

Accordingly, this example shows that the engineered synthetic pathway can support titers of at least ˜3 g/L thiamine from added precursors (module 3).

### REFERENCES

D1 Radical S-adenosylmethionine enzymes. Broderick J B, Duffus B R, Duschene K S, Shepard E M. Chem Rev. 2014 Apr. 23; 114(8):4229-317. doi: 10.1021/cr4004709.
D2 Glycine oxidase from B. subtilis: role of histidine 244 and methionine 261. Boselli A, Rosini E, Marcone G L, Sacchi S, Motteran L, Pilone M S, Pollegioni L, Molla G. Biochimie. 2007 November; 89(11):1372-80. doi: 10.1016/j.biochi.2007.04.019.
D3 Purification and Properties of Glycine Oxidase from P. putida KT2440. Equar M Y, Tani Y, Mihara H. J Nutr Sci Vitaminol (Tokyo). 2015; 61(6):506-10. doi: 10.3177/jnsv.61.506.
D4 Thiamine biosynthesis in Escherichia coli: isolation and initial characterisation of the ThiGH complex. Leonardi R, Fairhurst S A, Kriek M, Lowe D J, Roach P L. FEBS Lett. 2003 Mar. 27; 539(1-3):95-9. doi: 10.1016/s0014-5793(03)00204-7.
D5 Structure of the Escherichia coli ThiS-ThiF complex, a key component of the sulfur transfer system in thiamin biosynthesis. Lehmann C, Begley T P, Ealick S E. Biochemistry. 2006 Jan. 10; 45(1):11-9. doi: 10.1021/bi051502y.
D6 Thiamin biosynthesis in Escherichia coli. Identification of ThiS thiocarboxylate as the immediate sulfur donor in the thiazole formation. Taylor S V, Kelleher N L, Kinsland C, Chiu H J, Costello C A, Backstrom A D, McLafferty F W, Begley T P. J Biol Chem. 1998 J
D7 The rhodanese domain of ThiI is both necessary and sufficient for synthesis of the thiazole moiety of thiamine in Salmonella enterica. Martinez-Gomez N C, Palmer L D, Vivas E, Roach P L, Downs D M. J Bacteriol. 2011 September; 193(18):4582-7. doi: 10.1128/J B.05325-1
D8 U.S. Pat. No. 8,329,395 B1.
D9 (Microbial cell factories for the sustainable manufacturing of B vitamins. Carlos G. Acevedo-Rocha, Luisa S. Gronenberg, Matthias Mack, Fabian M. Commichau, Hans J. Genee. Cur. Opinion Biotech. 2019, Vol. 56, P 18-29).
D10 WO2017103221

1. A genetically modified host cell having improved production of thiamine, wherein the host cell expresses one or more heterologous ThiO enzymes converting glycine into dehydroglycine (DHG) in the host cell, whereby the production of the thiamine in the genetically modified host cell is improved compared to an unmodified parent host cell.

2. A genetically modified host cell having improved production of thiamine, wherein the host cell expresses one or more heterologous ThiI enzymes catalyzing the transfer of sulfur from IscS to the sulfur carrier protein ThiS in the host cell, whereby the production of the thiamine in the genetically modified host cell is improved compared to an unmodified parent host cell.

3. The host cell of claim 1, wherein the one or more heterologous ThiO enzymes is a bacterial ThiO enzyme.

4. The host cell of claim 2, wherein the one or more heterologous ThiI enzymes is a bacterial ThiI enzyme.

5. The host cell of claims 1 or 3, wherein the one or more heterologous bacterial ThiO enzymes is a BsThiO enzyme from Bacillus subtilis and/or a PpThiO enzyme from Pseudomonas putida.

6. The host cell of any preceding claim wherein one or more heterologous:
a) ThiO enzymes is at least 70%, such as at least 80%, such as at least 90%, such as at least 95%, such as at least 96%, such as at least 97%, such as at least 98%, such as at least 99%, such as 100% identical to the ThiO enzyme comprised in SEQ ID NO: 27 and/or 29; and/or
b) ThiI enzymes is at least 70%, such as at least 80%, such as at least 90%, such as at least 95%, such as at least 96%, such as at least 97%, such as at least 98%, such as at least 99%, such as 100% identical to the ThiI enzyme comprised in SEQ ID NO: 23, 35, 37, 39 and/or 41.

7. The host cell of claims 2 or 4, wherein the one or more bacterial ThiI enzymes is a E. coli ThiI enzyme having SEQ ID NO: 23

8. The host cell of claims 3 or 5, wherein the one or more heterologous bacterial ThiO enzymes is a BsThiO enzyme from Bacillus subtilis having SEQ ID NO: 27 and/or a PpThiO enzyme from Pseudomonas putida having SEQ ID NO: 29.

9. The host cell of any preceding claim, wherein the gene encoding:
a) the ThiO enzyme is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 28 or 30, or genomic DNA thereof encoding the ThiO enzyme comprised in SEQ ID NO: 27, and 29;
b) the ThiI enzyme is least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 24, 36, 38, 40 and/or 42 or genomic DNA thereof encoding the ThiI enzyme comprised in SEQ ID NO: 23, 35, 37, 39 and/or 41.

10. The host cell of any preceding claim, wherein the
a) BsThiO gene is at least 70%, such as at least 80%, such as at least 90%, such as at least 95%, such as at least 96%, such as at least 97%, such as at least 98%, such as at least 99%, such as 100% identical to the BsThiO gene comprised in SEQ ID NO: 28;
b) PpThiO gene is at least 70%, such as at least 80%, such as at least 90%, such as at least 95%, such as at least 96%, such as at least 97%, such as at least 98%, such as at least 99%, such as 100% identical to the PpThiO gene comprised in SEQ ID NO: 30.

11. The host cell of claims 7 or 9, wherein the ThiI gene is a truncated ThiI gene.

12. The host cell of claim 11, wherein the ThiI gene is a truncated ThiI comprising a rhodanese domain that transfers the sulfur from persulphide IscS to the sulfur carrier protein ThiS, forming ThiS-thiocarboxylate in the thiamine biosynthesis pathway.

13. The host cell of claim 12, wherein the rhodanese domain is at least 70%, such as at least 80%, such as at least 90%, such as at least 95%, such as at least 96%, such as at least 97%, such as at least 98%, such as at least 99%, such as 100% identical to the rhodanese domain comprised in SEQ ID NO: 13.

14. The host cell of any preceding claim, further comprising an operative metabolic pathway comprising one or more native or heterologous pathway elements producing the thiamine.

15. The host cell of claim 14, wherein one or more pathway elements comprise one or more radical SAM enzymes.

16. The host cell any preceding claim, wherein one or more pathway elements are selected from:
a) one or more phosphate synthase enzymes selected from phosphomethylpyrimidine synthase (ThiC); that catalyzes the synthesis of the hydroxymethylpyrimidine phosphate (HMP-P) moiety of thiamine from aminoimidazole ribotide (AIR) in a radical S-adenosyl-L-methionine (SAM)-dependent reaction;
b) a hydroxymethylpyrimidine/phosphomethylpyrimidine kinase (ThiD) that catalyzes the phosphorylation of hydroxymethylpyrimidine phosphate (HMP-P) to HMP-PP, and of HMP to HMP-P;
c) an adenylyltransferase (ThiF) that catalyzes the adenylation of the carboxy terminus of the sulfur carrier protein ThiS and the subsequent displacement of AMP catalyzed by ThiI-persulfide to give a ThiS-ThiI acyl disulfide ThiS;
d) a sulfur carrier protein (ThiS) in which its C-terminal thiocarboxylation occurs in 2 steps: First, it is acyl-adenlyated by ThiF and then thiocarboxylated by ThiI;
e) a 2-iminoacetate synthase (ThiH) converting that catalyzes the radical-mediated cleavage of tyrosine to 2-iminoacetate and 4-cresol;
f) a thiazole synthase (ThiG) that catalyzes the rearrangement of 1-deoxy-D-xylulose 5-phosphate (DXP) to produce the thiazole phosphate moiety of thiamine;
g) a hydroxyethylthiazole kinase (ThiM) that catalyzes the rearrangement of 1-deoxy-D-xylulose 5-phosphate (DXP) to produce the thiazole phosphate moiety of thiamine;
h) a thiamine mono-phosphate (TMP) phosphatase that dephosphorylate thiamine monophosphate to thiamine;
i) a thiamine kinase (ThiK) that catalyzes the phosphorylation of thiamine to thiamine phosphate;
j) a thiamine-monophosphate kinase (ThiL) which catalyzes the ATP-dependent phosphorylation of thiamine-monophosphate (TMP) to form thiamine-pyrophosphate (TPP);
k) a thiamine-phosphate synthase (ThiE) that condenses 4-methyl-5-(beta-hydroxyethyl)thiazole monophosphate (THZ-P) and 2-methyl-4-amino-5-hydroxymethyl pyrimidine pyrophosphate (HMP-PP) to form thiamine monophosphate (TMP)
l) an HTH-type transcriptional regulator (IscR) that regulates the transcription of several operons and genes involved in the biogenesis of Fe-S clusters and Fe-S-containing proteins.

17. The host cell of claims 14 to 16, wherein one or more pathway elements are encoded by one or more genes selected from the group of:
a) ThiC that has at least 70%, such at least 75%, such as at least 80%, such as at least 90%, such as at least 95%, such as at least 99%, such as 100% identity to the ThiC comprised in SEQ ID NO: 2;
b) ThiD that has at least 70%, such at least 75%, such as at least 80%, such as at least 90%, such as at least 95%, such as at least 99%, such as 100% identity to the ThiD comprised in SEQ ID NO: 4;
c) ThiF that has at least 70%, such at least 75%, such as at least 80%, such as at least 90%, such as at least 95%, such as at least 99%, such as 100% identity to the ThiF comprised in SEQ ID NO: 6;
d) ThiS has at least 70%, such at least 75%, such as at least 80%, such as at least 90%, such as at least 95%, such as at least 99%, such as 100% identity to the ThiS comprised in SEQ ID NO: 8;
e) ThiH that has at least 70%, such at least 75%, such as at least 80%, such as at least 90%, such as at least 95%, such as at least 99%, such as 100% identity to the ThiH comprised in SEQ ID NO: 10;
f) ThiG that has at least 70%, such at least 75%, such as at least 80%, such as at least 90%, such as at least 95%, such as at least 99%, such as 100% identity to the ThiG comprised in SEQ ID NO: 12;
g) ThiM that has at least 70%, such at least 75%, such as at least 80%, such as at least 90%, such as at least 95%, such as at least 99%, such as 100% identity to the ThiM comprised in SEQ ID NO: 14;
h) TMP phosphatase that has at least 70%, such at least 75%, such as at least 80%, such as at least 90%, such as at least 95%, such as at least 99%, such as 100% identity to the TMP phosphatase comprised in SEQ ID NO: 16;
i) ThiK that has at least 70%, such at least 75%, such as at least 80%, such as at least 90%, such as at least 95%, such as at least 99%, such as 100% identity to the Thik comprised in SEQ ID NO: 18;
j) ThiL that has at least 70%, such at least 75%, such as at least 80%, such as at least 90%, such as at least 95%, such as at least 99%, such as 100% identity to the ThiL comprised in SEQ ID NO: 20;
k) ThiE that has at least 70%, such at least 75%, such as at least 80%, such as at least 90%, such as at least 95%, such as at least 99%, such as 100% identity to the ThiE comprised in SEQ ID NO: 22;
l) IscR that has at least 70%, such at least 75%, such as at least 80%, such as at least 90%, such as at least 95%, such as at least 99%, such as 100% identity to the IscR comprised in SEQ ID NO: 32.

18. The host cell of any preceding claim, wherein the endogenous native ThiH gene is disrupted or inactivated.

19. The host cell of claim 18, wherein the disruption/inactivation is caused by introducing a deletion through complete removal of the gene or a translational knockout by introducing one or more stop codons or frameshift mutations preventing expression of an active peptide.

20. The host cell of claims 18 or 19, wherein the disruption/inactivation comprises a translational knockout or a frameshift mutation.

21. The host cell of claims 18 to 20, wherein the deletion, disruption and/or attenuation is a point mutation in a promoter for the protein-encoding sequence, in the RBS region and/or in a protein-encoding sequence.

22. The host cell of any preceding claim, wherein one or more pathway elements are encoded by one or more genes selected from the group of:
a) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 2 or genomic DNA thereof encoding the ThiC enzyme comprised in SEQ ID NO: 1;
b) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 4 or genomic DNA thereof encoding the ThiD enzyme comprised in SEQ ID NO: 3;
c) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 6 or genomic DNA thereof encoding the ThiF enzyme comprised in SEQ ID NO: 5;
d) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 8 or genomic DNA thereof encoding the ThiS enzyme comprised in SEQ ID NO: 7;
e) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 10 or genomic DNA thereof encoding the ThiH enzyme comprised in SEQ ID NO: 9;
f) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 12 or genomic DNA thereof encoding the ThiG enzyme comprised in SEQ ID NO: 11;
g) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 14 or genomic DNA thereof encoding the ThiM enzyme comprised in SEQ ID NO: 13;
h) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 16 or genomic DNA thereof encoding the TMP enzyme phosphatase comprised in SEQ ID NO: 15;
i) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 18, or genomic DNA thereof encoding the ThiK enzyme comprised in SEQ ID NO: 17;
j) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 20, or genomic DNA thereof encoding the ThiL enzyme comprised in SEQ ID NO: 19;
k) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 22, or genomic DNA thereof encoding the ThiE enzyme comprised in SEQ ID NO: 21; and
l) a gene which is at least 50%, such as at least 60%, such as at least 70%, such as at least 80%, such as at least 90%, such as at least 95%, such as at least 96%, such as at least 97%, such as at least 98%, such as at least 99%, such as 100% identical to the gene comprised in SEQ ID NO: 32, or genomic DNA thereof encoding the IscR enzyme comprised in SEQ ID NO: 31.

23. The host cell of any preceding claim, wherein one or more genes and/or polypeptides of the pathway for the thiamine are heterologous to the host cell.

24. The host cell of any preceding claim, further comprising at least 2 copies of one or more genes and/or polypeptides of the pathway for the thiamine.

25. The host cell of any preceding claim, further comprising a transporter molecule facilitating transport of a precursor for or a product of the pathway for the thiamine.

26. The host cell of any preceding claim, wherein the host cell is further genetically modified to provide an increased amount of a substrate in the pathway for the thiamine.

27. The host cell of any preceding claim, wherein the host cell is further genetically modified to exhibit increased tolerance towards one or more substrates, intermediates, or products in the pathway for the thiamine.

28. The host cell of any preceding claim, wherein one or more additional native or endogenous genes of the host cell are deleted, disrupted and/or attenuated.

29. The host cell of any preceding claim, wherein one or more genes in the pathway for the thiamine are overexpressed.

30. The host cell of any preceding claim, wherein the host cell is prokaryotic or eukaryotic.

31. The host cell of any preceding claim, wherein the host cell is further genetically modified to provide an increased amount of a substrate in the pathway for the thiamine.

32. A cell culture comprising the genetically modified host cells of any preceding claim and a growth medium.

33. A method for producing a thiamine comprising
a) culturing the cell culture of claim 32 at conditions allowing the host cells to produce the thiamine; and
b) optionally recovering and/or isolating the thiamine.

34. The method of claim 33, further comprising feeding one or more exogenous thiamine precursors to the host cell culture.

35. The method of claim 34, wherein the feeding one or more exogenous thiamine precursors to the host cell culture, comprises feeding THZ, HMP or THZ to the cell culture.

36. The method of claims 33 to 35, further comprising one or more elements selected from:
a) culturing the cell culture under aerobic or anaerobic conditions
b) cultivating the host cells under mixing;
c) cultivating the host cells at a temperature of between 25° C. to 50° C.;
d) cultivating the host cells at a pH of between 3-9; and
e) cultivating the host cells for between 10 hours to 120 days.

37. The method of claims 33 to 36, wherein the recovering and/or isolation step comprises separating a liquid phase of the cell culture from a solid phase of the cell culture to obtain a supernatant comprising the thiamine and subjecting the supernatant to one or more steps selected from:
a) contacting the supernatant with one or more adsorbent resins in order to obtain at least a portion of the produced thiamine, then optionally recovering the thiamine from the resin in a concentrated solution prior to isolation of the thiamine by crystallisation or solvent evaporation;
b) contacting the supernatant with one or more ion exchange or reversed-phase chromatography columns to obtain at least a portion of the thiamine, then optionally recovering the thiamine from the resin in a concentrated solution prior to isolation of the thiamine by crystallisation or solvent evaporation; and/or
c) extracting the thiamine from the supernatant, such as by liquid-liquid extraction into an immiscible solvent, then optionally isolating the thiamine by crystallisation or solvent evaporation;
d) thereby recovering and/or isolating the thiamine.

38. The method of claims any preceding claims 33 to 37, wherein the thiamine yield is at least 10% higher such as at least 20%, such as at least 50%, such as least 100%, such as at least 200% higher than production by a host cell without
a) expressing one or more heterologous ThiO genes capable of converting glycine into dehydroglycine (DHG) in the host cell and/or,
b) over-expressing one or more ThiI genes capable of catalysing the transfer of sulfur to the sulfur carrier protein ThiS in the host cell.

39. The method of claim 38 wherein the thiamine yield and/or titer is at least 400 mg/L, optionally at least 800 mg/L, optionally at least 1000 mg/L, optionally at least 1500 mg/L, optionally at least 2000 mg/L, optionally at least 3000 mg/L, optionally at least 5000 mg/L, optionally at least 10000 mg/L, optionally at least 15000 mg/L, optionally at least 20000 mg/L.

40. The method of claims 33 to 39, wherein one or more steps of producing the thiamine is performed in vitro.

41. The method of claims 33 to 40, further comprises mixing the thiamine with one or more carriers, agents, adjuvants, additives and/or excipients, optionally pharmaceutical grade carriers, agents, adjuvants, additives and/or excipients.

42. A fermentation composition comprising the cell culture of claim 32 or resulting from the method of any of claims 33 to 41 and the thiamine.

43. The fermentation composition of claim 42, wherein at least 50%, such as at least 75%, such as at least 95%, such as at least 99% of the genetically modified host cells are lysed and/or disintegrated.

44. The fermentation composition of claim 42 or 43, wherein at least 50%, such as at least 75%, such as at least 95%, such as at least 99% of solid cellular material has separated from the composition.

45. The fermentation composition of claims 42 to 44, further comprising one or more compounds selected from:
a) precursor or products of the operative metabolic pathway producing the thiamine;
b) supplemental nutrients; and
c) wherein the concentration of the thiamine is at least 1 mg/L composition.

46. A composition comprising the fermentation composition of claims 42 to 45 and one or more carriers, agents, adjuvants, additives and/or excipients.

47. The composition of claim 46, wherein the fermentation composition and the one or more carriers, agents, adjuvants, additives and/or excipients are in a dry solid form.

48. The composition of claims 46 or 47, wherein the fermentation composition and the one or more carriers, agents, adjuvants, additives and/or excipients are in a liquid stabilized form.

49. The composition of claims 46 to 48, wherein the fermentation composition is refined into a pharmaceutical preparation, a dietary supplement, a cosmetic, a food preparation, a feed preparation and/or an analytical or diagnostic reagent.

50. The composition of claim 49, wherein the pharmaceutical preparation is in form of a powder, tablet, capsule, hard chewable and or soft lozenge or a gum.

51. The composition of claims 49 or 50, wherein the pharmaceutical preparation is in form of a liquid pharmaceutical solution.

52. The composition of claims 46 to 51 for use as a medicament.

53. The composition of claims 46 to 52 for use in the treatment of a nutritional deficiency.

54. A method for treating a disease in a mammal, comprising administering a therapeutically effective amount of the composition of claims 46 to 53 to the mammal.

55. The method of claim 54, wherein the disease is a nutritional deficiency or associated with a nutritional deficiency.


SEQUENCE LISTING (48 sequences)

SEQ ID NO: 1 - protein, 631 aa, Escherichia coli
MSATKLTRREQRARAQHFIDTLEGTAFPNSKRIYITGTHPGVRVPMREIQLSPTLIGGSKEQPQYEENEAIPVYDTSGPYGDPQIAINVQQGLAKLRQPWIDARGDTEELTVRSSDYTKARLADDGLDELRFSGVLTPKRAKAGRRVTQLHYARQGIITPEMEFIAIRENMGRERIRSEVLRHQHPGMSFGAHLPENITAEFVRDEVAAGRAIIPANINHPESEPMIIGRNFLVKVNANIGNSAVTSSIEEEVEKLVWSTRWGADTVMDLSTGRYIHETREWILRNSPVPIGTVPIYQALEKVNGIAEDLTWEAFRDTLLEQAEQGVDYFTIHAGVLLRYVPMTAKRLTGIVSRGGSIMAKWCLSHHQENFLYQHFREICEICAAYDVSLSLGDGLRPGSIQDANDEAQFAELHTLGELTKIAWEYDVQVMIEGPGHVPMQMIRRNMTEELEHCHEAPFYTLGPLTTDIAPGYDHFTSGIGAAMIGWFGCAMLCYVTPKEHLGLPNKEDVKQGLITYKIAAHAADLAKGHPGAQIRDNAMSKARFEFRWEDQFNLALDPFTARAYHDETLPQESGKVAHFCSMCGPKFCSMKISQEVRDYAATQTIEMGMADMSENFRARGGEIYLRKEEA

SEQ ID NO: 2 - DNA, 1884 bp, Escherichia coli
  source 1..1884: mol_type = other DNA
atgtctgcaacaaaactgacccgccgcgaacaacgcgcccgggcccaacattttatcgacaccctggaaggcaccgcctttcccaactcaaaacgcatttatatcactggcacacaccccggcgtgcgcgtgccgatgcgtgagatccagcttagcccgacgctaattggcggtagcaaagaacagccgcagtacgaagaaaacgaagcgattccggtctacgacacctccggcccgtatggtgatccgcagattgccattaacgtgcagcaagggctggcaaaactacgccagccgtggatcgatgcgcgcggcgataccgaagaacttaccgtgcgcagttccgattacactaaagcgcggctggcagatgatggcctcgacgaactgcgttttagcggcgtactaacaccaaaacgcgccaaagcaggacgccgtgtcacccaactgcactacgcccgccagggcatcatcacgccggaaatggaattcatcgccatccgcgagaatatgggccgcgagcgcatccgtagcgaggttttacgccaccagcatccgggaatgagctttggcgcacatctgccggaaaatatcactgcggaatttgtccgtgatgaagttgctgccggacgtgcgattatcccggccaacattaatcatccggaatcggagccgatgattattggtcgcaatttcctggtaaaagttaacgccaatatcggcaactcggcggtcacctcttccatcgaagaagaagtggaaaagctggtatggtccacgcgctggggagcggatacggtgatggatctctccaccggtcgctatattcacgaaacccgcgagtggattttgcgtaacagcccggtgccgatcggtacagtgccgatctaccaggcgctggagaaggttaacgggatcgccgaagatcttacctgggaagcgttccgcgacacgctgctggaacaggccgagcaaggtgtggattacttcactatccatgcgggcgtactgctgcgctatgtgccgatgaccgcgaaacgcctgaccggtatcgtctctcgcggcggttcgattatggcgaaatggtgcctctcccatcatcaggaaaatttcctctatcaacacttccgcgaaatttgtgaaatctgtgccgcttatgacgtttcgctgtcgctgggcgacggtctgcgccccggttctattcaggacgccaacgatgaagcgcagtttgccgagctgcatacgctgggcgaactgaccaaaattgcctgggaatatgacgtgcaggtgatgattgaaggcccaggccacgtgccgatgcagatgatccgccgcaatatgaccgaggagttagagcactgccacgaagcgccgttttacactctggggccgctaactaccgatattgcgccgggctatgaccacttcacgtcggggattggtgcggcgatgattggctggtttggctgcgcgatgctctgttacgtaacgccaaaagagcatctgggtctgcccaataaagaagatgttaagcaggggcttatcacctataagattgctgcccacgccgctgacctggcgaaagggcatccgggcgcgcaaattcgcgataacgccatgtcgaaagcccgcttcgaatttcgctgggaagaccagtttaatctggccctcgacccgtttaccgcccgcgcttatcacgatgaaaccctgccgcaagagtcaggtaaagtcgcccatttttgctccatgtgtgggccgaaattctgctcgatgaaaatcagccaggaagtgcgtgattacgccgccacgcaaactattgaaatgggaatggcggatatgtcggagaacttccgtgccagaggcgggagaaatctacctgcgtaa

SEQ ID NO: 3 - protein, 266 aa, Escherichia coli
MKRINALTIAGTDPSGGAGIQADLKTFSALGAYGCSVITALVAQNTRGVQSVYRIEPDFVAAQLDSVFSDVRIDTTKIGMLAETDIVEAVAERLQRYQIQNVVLDTVMLAKSGDPLLSPSAVATLRSRLLPQVSLITPNLPEAAALLDAPHARTEQEMLEQGRSLLAMGCGAVLMKGGHLDDEQSPDWLFTREGEQRFTAPRIMTKNTHGTGCTLSAALAALRPRHTNWADTVQEAKSWLSSALAQADTLEVGHGIGPVHHFHAWW

SEQ ID NO: 4 - DNA, 801 bp, Escherichia coli
  source 1..801: mol_type = other DNA
atgaaacgaattaacgctctgacgattgccggtactgatccgagtggtggtgcggggattcaggccgatcttaaaaccttctcggcacttggcgcttatggttgctcagttattactgcactggtggcgcaaaatacccgtggcgtacagtcggtgtatcgcattgagcctgattttgtcgccgcccagctcgattcggtgttcagcgatgtgcgaatcgataccactaaaatcggtatgctggcggaaaccgatattgttgaagcggtggcagaacggttgcaacgttatcagatccaaaacgtggtactcgacaccgttatgctggcaaaaagcggcgacccgctgctttcaccttcggcggttgctacgctgcgcagtcgattattgccacaggtttcattaataacgccaaacttgcccgaagctgccgccttgctcgacgcgccacacgcgcgcaccgaacaggaaatgctggaacaagggcgatcgctgttggcgatgggctgtggcgcagtgctaatgaaaggtggtcatctggatgatgagcaaagcccggactggctgtttacccgcgagggtgaacaacggtttaccgcaccgcgcattatgaccaaaaacacccacggcactggttgtacactctctgcggcgttggctgcactacgcccgcgccatacaaactgggctgacaccgtacaggaggcaaaaagctggctttcatcggcgttagcccaggccgacacgctggaagttggtcacggtattggtccggttcaccacttccacgcctggtggtga

SEQ ID NO: 5 - protein, 251 aa, Escherichia coli
MNDRDFMRYSRQILLDDIALDGQQKLLDSQVLIIGLGGLGTPAALYLAGAGVGTLVLADDDDVHLSNLQRQILFTTEDIDRPKSQVSQQRLTQLNPDIQLTALQQRLTGEALKDAVARADVVLDCTDNMATRQEINAACVALNTPLITASAVGFGGQLMVLTPPWEQGCYRCLWPDNQEPERNCRTAGVVGPVVGVMGTLQALEAIKLLSGIETPAGELRLFDGKSSQWRSLALRRASGCPVCGGSNADPV

SEQ ID NO: 6 - DNA, 753 bp, Escherichia coli
  source 1..753: mol_type = other DNA
atgaatgaccgtgactttatgcgttatagccgccaaatcctgctcgacgatatcgctctggacgggcagcaaaaactgctcgacagccaggtgctgattatcggtctgggcgggctgggtacacctgctgcgctgtacctggcgggcgctggcgtcgggacgctggtactggcagatgacgacgatgtgcatttaagcaatctgcaacgacaaatcctctttaccactgaagatatcgatcgcccgaaatcgcaggtcagccaacagcgactgacacagttgaatcccgacattcaactgacagcattacaacaacggttaacgggtgaggcgttaaaagatgcggttgcacgggccgatgtggtgctcgactgtaccgacaatatggcgactcgccaggagattaatgccgcctgcgtggcactcaacacgccgcttatcaccgccagcgcggtcggatttggcggtcagttgatggtactgacgccgccctgggagcaggggtgttaccgctgcctgtggccagataaccaggagccagaacgcaactgccgcacggcgggcgtggttggcccggtggtcggggttatgggcactttgcaggcactggaagccattaagttattaagcggtatagagacacctgcgggagaactccgactgttcgacggtaaatcgagccagtgcagcctggcgttgcgccgcgccagtggttgcccggtatgcggaggaagcaatgcagatcctgtttaa

SEQ ID NO: 7 - protein, 66 aa, Escherichia coli
MQILFNDQAMQCAAGQTVHELLEQLDQRQAGAALAINQQIVPREQWAQHIVQDGDQILLFQVIAGG

SEQ ID NO: 8 - DNA, 201 bp, Escherichia coli
  source 1..201: mol_type = other DNA
atgcagatcctgtttaacgatcaagcgatgcagtgcgccgccgggcaaactgttcacgaactactggagcaactcgaccaacgacaagcgggcgcggccctggcgattaatcagcaaatcgtcccgcgtgagcagtgggcgcaacatatcgtgcaggatggcgaccagatcctgctttttcaggttattgcagggggttga

SEQ ID NO: 9 - protein, 377 aa, Escherichia coli
MKTFSDRWRQLDWDDIRLRINGKTAADVERALNASQLTRDDMMALLSPAASGYLEQLAQRAQRLTRQRFGNTVSFYVPLYLSNLCANDCTYCGFSMSNRIKRKTLDEADIARESAAIREMGFEHLLLVTGEHQAKVGMDYFRRHLPALREQFSSLQMEVQPLAETEYAELKQLGLDGVMVYQETYHEATYARHHLKGKKQDFFWRLETPDRLGRAGIDKIGLGALIGLSDNWRVDSYMVAEHLLWLQQHYWQSRYSVSFPRLRPCTGGIEPASIMDERQLVQTICAFRLLAPEIELSLSTRESPWFRDRVIPLAINNVSAFSKTQPGGYADNHPELEQFSPHDDRRPEAVAAALTAQGLQPVWKDWDSYLGRASQRL

SEQ ID NO: 10 - DNA, 1134 bp, Escherichia coli
  source 1..1134: mol_type = other DNA
atgaaaaccttcagcgatcgctggcgacaactggactgggacgacatccgcctgcgtatcaacggcaaaacggctgctgacgtagagcgggcgctaaatgcctcgcaactcacccgcgacgacatgatggcgctgttatcgcctgccgccagtggctatctggaacaactggcccaacgggcgcagcgtctgacccgtcagcgatttggcaacacagttagtttctacgtcccgctttatctttccaatctttgcgctaacgactgcacgtactgtggattttccatgagtaatcgcatcaagcgcaaaacgctggatgaagcggatattgccagggaaagtgccgctatacgggagatgggctttgaacatctgctgttagtcactggtgaacatcaggcgaaagtggggatggattactttcgtcgtcatctccctgcccttcgtgaacagttctcttcactacagatggaagtgcaaccgctggcggagacggaatacgccgagttaaagcaacttggtctggatggcgtgatggtttatcaggagacatatcacgaggcgacttatgcccgccatcatctgaaaggcaaaaaacaggacttcttctggcggctggaaaccccggatcggctggggcgtgcggggattgataagataggcctcggcgcgctaattggcctttccgacaactggcgcgttgacagctatatggttgccgaacatttgctatggctgcaacagcattactggcaaagccgttactctgtctcctttccgcgcctgcgcccgtgtactggcggcattgagcctgcgtcgattatggatgaacgccagttagtgcaaaccatctgcgccttccgactgcttgcaccggagattgaactgtcactctccacgcgggaatcaccgtggtttcgcgatcgcgttattccgctggcgatcaataacgtcagcgccttctcgaaaacgcagccaggtggctatgccgataatcaccccgagttggaacagttctcaccgcacgacgatcgcagaccggaagcggttgctgccgcgttaaccgctcagggtttgcagccggtatggaaagactgggacagctatctgggacgcgcctcgcaaagactatga

SEQ ID NO: 11 - protein, 256 aa, Escherichia coli
MLRIADKTFDSHLFTGTGKFASSQLMVEAIRASGSQLVTLAMKRVDLRQHNDAILEPLIAAGVTLLPNTSGAKTAEEAIFAAHLAREALGTNWLKLEIHPDARWLLPDPIETLKAAETLVQQGFVVLPYCGADPVLCKRLEEVGCAAVMPLGAPIGSNQGLETRAMLEIIIQQATVPVVVDAGIGVPSHAAQALEMGADAVLVNTAIAVADDPVNMAKAFRLAVEAGLLARQSGPGSRSYFAHATSPLTGFLEASA

SEQ ID NO: 12 - DNA, 771 bp, Escherichia coli
  source 1..771: mol_type = other DNA
atgttacgtattgcggacaaaacgtttgattcacatctgtttaccggcacagggaaattcgcttcttcacaactgatggtggaggcgatccgcgcttccggcagccagctggtgacactggcgatgaaacgtgtcgacttgcgccagcacaacgacgctatcctcgaaccgcttatcgcggcgggtgtgaccctgctgccaaatacatccggggcgaaaacagcggaagaagccattttcgccgcccatctggctcgtgaagcgttaggcacaaactggttaaaattagagattcaccctgacgcccgctggctgttgcccgatcccatcgaaaccctgaaagccgccgaaacgctggtacaacagggatttgtcgtgctgccttactgcggggccgatccggtattgtgtaaacgtctggaagaagtcggctgtgcagcggtgatgccgctcggcgcgccgattggctcgaatcagggactggaaacccgcgccatgctggagattattatccagcaggccacagtgccggtggttgtcgatgctggcatcggcgttcccagccatgccgcgcaggcgctggaaatgggggccgacgcggtgttagtgaatacggcgattgccgtcgcggacgatcccgtcaacatggcgaaggcatttcgtctggcggtagaagcaggcctactggcacgtcagtccggaccgggcagccgcagttattttgctcatgccaccagcccgctgaccggatttctggaggcatcggcatga

SEQ ID NO: 13 - protein, 262 aa, Escherichia coli
MQVDLLGSAQSAHALHLFHQHSPLVHCMTNDVVQTFTANTLLALGASPAMVIETEEASQFAAIASALLINVGTLTQPRAQAMRAAVEQAKSSQTPWTLDPVAVGALDYRRHFCHELLSFKPAAIRGNASEIMALAGIANGGRGVDTTDAAANAIPAAQTLARETGAIVVVTGEMDYVTDGHRIIGIHGGDPLMTKVVGTGCALSAVVAACCALPGDTLENVASACHWMKQAGERAVARSEGPGSFVPHFLDALWQLTQEVQA

SEQ ID NO: 14 - DNA, 789 bp, Escherichia coli
  source 1..789: mol_type = other DNA
atgcaagtcgacctgctgggttcagcgcaatctgcgcacgcgttacacctttttcaccaacattcccctcttgtgcactgcatgaccaatgatgtggtgcaaacctttaccgccaataccttgctggcgctcggtgcatcgccagcgatggttatcgaaaccgaagaggccagtcagtttgcggctatcgccagtgccttgttgattaacgttggcacactgacgcagccacgcgctcaggcgatgcgtgctgccgttgagcaagcaaaaagctctcaaacaccctggacgcttgatccagtagcggtgggtgcgctcgattatcgccgccatttttgtcatgaacttttatcttttaaaccggcagcgatacgtggtaatgcttcggaaatcatggcattagctggcattgctaatggcggacggggagtggataccactgacgccgcagctaacgcgatacccgctgcacaaacactggcacgggaaactggcgcaatcgtcgtggtcactggcgagatggattatgttaccgatggacatcgtatcattggtattcacggtggtgatccgttaatgaccaaagtggtaggaactggctgtgcattatcggcggttgtcgctgcctgctgtgcgttaccaggcgatacgctggaaaatgtcgcatctgcctgtcactggatgaaacaagccggagaacgcgcagtcgccagaagcgaggggccaggcagttttgttccacatttccttgatgcgctctggcaattgacgcaggaggtgcaggcatga

SEQ ID NO: 15 - protein, 617 aa, Arabidopsis thaliana
MRFLFPTRLINNSSLGLLRSPHTTAPIRSLWFRTKSPVFRSATTPIMTAVAFSSSLSIPPTSEEALPGKLWIKFNRECLFSIYSPFAVCLAAGNLKIDTFRQYIAQDVHFLKAFAHAYELAADCADDDDDKLAISDLRKSVMEELKMHDSFVQDWDLDINKEVSVNSATLRYTEFLLATASGKVEGCKAPGMLDTPFEKTKVAAYTLGAVTPCMRLYAFLGKEFGSLLDLSDVNHPYKKWIDNYSSDAFQASAKQTEDLLEKLSVSMTGEELDIIEKLYQQAMKLEVEFFHAQPLAQPTIVPLLKNHSKDDLVIFSDFDLTCTVVDSSAILAEIAIVTAPKDEQSRSGQQIHRMLSSDLKNTWNLLSKQYTEHYEECIESILNKKKADKFDYEGLCKALEQLSDFEKEANNRVIESGVLKGLNLEDIKRAGERLILQDGCINVFQKILKTENLNAELHVLSYCWCGDLIRAAFSAGGVDAVEVHANEFTFEESISTGEIERKVESPINKAQQFKSILQNRKNENNKKSFLSVYIGDSVGDLLCLLEADIGIVVSSSSSLRRVGSHFGVSFVPLFSGIVQKQKQHTEESSSSAWKGLSGTLYTVSSWAEIHSFALGWE

SEQ ID NO: 16 - DNA, 801 bp, Arabidopsis thaliana; identical to SEQ ID NO: 4
  source 1..801: mol_type = other DNA

SEQ ID NO: 17 - protein, 274 aa, Escherichia coli
MPFRSNNPITRDELLSRFFPQYHPVTTFNSGLSGGSFLIEHQGQRFVVRQPHDPDAPQSAFLRQYRALSQLPACIAPKPHLYLRDWMVVDYLPGAVKTYLPDTNELAGLLYYLHQQPRFGWRITLLPLLELYWQQSDPARRTVGWLRMLKRLRKAREPRPLRLSPLHMDVHAGNLVHSASGLKLIDWEYAGDGDIALELAAVWVENTEQHRQLVNDYATRAKIYPAQLWRQVRRWFPWLLMLKAGWFEYRWRQTGDQQFIRLADDTWRQLLIKQ

SEQ ID NO: 18 - DNA, 825 bp, Escherichia coli
  source 1..825: mol_type = other DNA
gtgccgtttcgcagcaataatcccatcacgcgcgacgaattgctgtcgcgctttttcccgcagtatcatcccgtcacgacgtttaatagtgggcttagtggcgggagttttctcattgaacatcagggccagcgttttgttgtgcgtcagccgcacgatcctgatgcgccgcagtccgcgttcttgcgccagtatcgggctttatcacaactacccgcatgcattgcaccgaagccgcatttatatctccgtgactggatggtagtcgactatctgcccggcgcggtaaaaacgtatttgccggataccaacgaactggcaggcttgctgtattatctacatcaacaaccacgttttggctggcgaataacgctgttgccgttactggaactgtactggcagcaaagcgatccggcgcggcggacagtgggttggctgcgaatgttaaaacgtctgcgcaaagcgcgggaaccacggcctttacgcttaagtccattgcatatggatgtccacgccggaaatttagtgcatagcgcgtcagggttaaaactcatcgactgggagtatgccggagatggtgatatcgcgctggaactggcggcggtgtgggtggaaaatactgaacagcaccggcaattggtcaatgactatgccactcgcgcgaagatttatccggcgcaattatggcgtcaggtcaggcgatggtttccctggctgctgatgctcaaagcagggtggtttgagtaccgctggcgacaaaccggcgatcaacaatttatcaggctggccgatgacacctggcggcagctattaataaaacaataa

SEQ ID NO: 19 - protein, 325 aa, Escherichia coli
MACGEFSLIARYFDRVRSSRLDVELGIGDDCALLNIPEKQTLAISTDTLVAGNHFLPDIDPADLAYKALAVNLSDLAAMGADPAWLTLALTLPDVDEAWLESFSDSLFDLLNYYDMQLIGGDTTRGPLSMTLGIHGFVPMGRALTRSGAKPGDWIYVTGTPGDSAAGLAILQNRLQVADAKDADYLIKRHLRPSPRILQGQALRDLANSAIDLSDGLISDLGHIVKASDCGARIDLALLPFSDALSRHVEPEQALRWALSGGEDYELCFTVPELNRGALDVALGHLGVPFTCIGQMTADIEGLCFIRDGEPVTLDWKGYDHFATP

SEQ ID NO: 20 - DNA, 978 bp, Escherichia coli
  source 1..978: mol_type = other DNA
atggcatgtggcgagttctccctgattgcccgttattttgaccgtgtaagaagttctcgtcttgatgtcgaactgggcatcggcgacgattgcgcacttctcaatatccccgagaaacagaccctggcgatcagcactgatacgctggtggcgggtaaccatttcctccctgatatcgatcctgctgatctggcttataaagcactggcggtgaacctaagcgatctggcagcgatgggggccgatccggcctggctgacgctggcattaaccttaccggacgtagacgaagcgtggcttgagtccttcagcgacagtttgtttgatcttctcaattattacgatatgcaactcattggcggcgataccacgcgtgggccattatcaatgacgttgggtatccacggctttgttccgatgggacgagccttaacgcgctctggggcgaaaccgggtgactggatctatgtgaccggtacaccgggcgatagcgccgccgggctggcgattttgcaaaaccgtttgcaggttgccgatgctaaagatgcggactacttgatcaaacgtcatctccgtccatcgccgcgtattttacaggggcaggcactgcgcgatctggcaaattcagccatcgatctctctgacggtttgatttccgatctcgggcatatcgtgaaagccagcgactgcggcgcacgtattgacctggcattgctgccgttttctgatgcgctttctcgccatgttgaaccggaacaggagctgcgctgggcgctctctggcggtgaagattacgagttgtgtttcactgtgccggaactgaaccgtggcgcgctggatgtggctctcggacacctgggcgtaccgtttacctgtatcgggcaaatgaccgccgatatcgaagggctttgttttattcgtgacggcgaacctgttacattagactggaaaggatatgaccattttgccacgccataa

SEQ ID NO: 21 - protein, 211 aa, Escherichia coli
MYQPDFPPVPFRSGLYPVVDSVQWIERLLDAGVRTLQLRIKDRRDEEVEADVVAAIALGRRYNARLFINDYWRLAIKHQAYGVHLGQEDLQATDLNAIRAAGLRLGVSTHDDMEIDVALAARPSYIALGHVFPTQTKQMPSAPQGLEQLARHVERLADYPTVAIGGISLARAPAVIATGVGSIAVVSAITQAADWRLATAQLLEIAGVGDE

SEQ ID NO: 22 - DNA, 636 bp, Escherichia coli
  source 1..636: mol_type = other DNA
atgtatcagcctgattttcctcctgtaccttttcgttcaggactgtacccggtggtggacagcgtacagtggatcgaacgtctgttggatgcaggcgtacgtactctccagctacgcatcaaagatcggcgcgatgaagaggtggaagccgatgtcgtggcggcaattgcgctgggccgccgctataacgcgcgattgtttatcaacgattactggcggctggcgatcaagcatcaggcgtatggcgtccatttggggcaggaagatttgcaagccaccgatctcaatgccatccgcgcggcaggcctgcggctgggcgtttcgacacatgacgatatggaaatcgacgtcgcgctggcagcacgcccctcttatatcgcgctgggacatgtgttcccgacgcaaaccaaacagatgccttctgcaccgcaggggctggaacagctggcacggcatgttgagcgactggcggattatcccaccgtggcgattggcggtatcagtctggcacgcgcgcctgcggtgatagcaacgggtgtcggcagtatcgccgtcgtcagcgccattactcaagccgcagactggcgtttggcaacggcacagttgctggaaattgcaggagttggcgatgaatga

SEQ ID NO: 23 - protein, 482 aa, Escherichia coli
MKFIIKLFPEITIKSQSVRLRFIKILTGNIRNVLKHYDETLAVVRHWDNIEVRAKDENQRLAIRDALTRIPGIHHILEVEDVPFTDMHDIFEKALVQYRDQLEGKTFCVRVKRRGKHDFSSIDVERYVGGGLNQHIESARVKLTNPDVTVHLEVEDDRLLLIKGRYEGIGGFPIGTQEDVLSLISGGFDSGVSSYMLMRRGCRVHYCFFNLGGAAHEIGVRQVAHYLWNRFGSSHRVRFVAINFEPVVGEILEKIDDGQMGVILKRMMVRAASKVAERYGVQALVTGEALGQVSSQTLTNLRLIDNVSDTLILRPLISYDKEHIINLARQIGTEDFARTMPEYCGVISKSPTVKAVKSKIEAEEEKFDFSILDKVVEEANNVDIREIAQQTEQEVVEVETVNGFGPNDVILDIRSIDEQEDKPLKVEGIDVVSLPFYKLSTKFGDLDQNKTWLLWCERGVMSRLQALYLREQGFNNVKVYRP

SEQ ID NO: 24 - DNA, 1449 bp, Escherichia coli
  source 1..1449: mol_type = other DNA
atgaagtttatcattaaattgttcccggaaatcaccatcaaaagccaatctgtgcgcttgcgctttataaaaatccttaccgggaacattcgtaacgttttaaagcactatgatgagacgctcgctgtcgtccgccactgggataacatcgaagttcgcgcaaaagatgaaaaccagcgtctggctattcgcgacgctctgacccgtattccgggtatccaccatattctcgaagtcgaagacgtgccgtttaccgacatgcacgatattttcgagaaagcgttggttcagtatcgcgatcagctggaaggcaaaaccttctgcgtacgcgtgaagcgccgtggcaaacatgattttagctcgattgatgtggaacgttacgtcggcggcggtttaaatcagcatattgaatccgcgcgcgtgaagctgaccaatccggatgtgactgtccatctggaagtggaagacgatcgtctcctgctgattaaaggccgctacgaaggtattggcggtttcccgatcggcacccaggaagatgtgctgtcgctcatttccggtggtttcgactccggtgtttccagttatatgttgatgcgtcgcggctgccgcgtgcattactgcttctttaacctcggcggcgcggcgcatgaaattggcgtgcgtcaggtggcgcattatctgtggaaccgctttggcagctcccaccgcgtgcgttttgtcgctattaatttcgaaccggtcgtcggggaaattctcgagaaaatcgacgacggtcagatgggcgttatcctcaaacgtatgatggtgcgtgccgcatctaaagtggctgaacgttacggcgtacaggcgctggtcaccggcgaagcgctcggccaggtgtccagccagacgctgaccaacctgcgcctgattgataacgtctccgacacgctgatcctgcgtccgctgatctcttacgacaaagagcacatcatcaacctggcccgccagattggcaccgaagactttgctcgcacgatgccggaatattgtggtgtgatctccaaaagcccgacggtgaaagcagttaaatcgaagattgaagcggaagaagagaagttcgacttcagcattctcgataaagtggttgaggaagcgaataacgttgatatccgcgaaatcgcccagcagaccgagcaggaagtggtggaagtggaaaccgtcaatggcttcggcccgaacgacgtgatcctcgatatccgttctatcgatgaacaggaagataagccactgaaagtcgaagggattgatgtggtttctctgccgttctataaactgagcaccaaatttggcgatctcgaccagaacaaaacctggctgctgtggtgtgagcgcggggtgatgagccgtctgcaggcgctctatctgcgcgagcagggctttaacaatgtgaaggtatatcgcccgtaa

SEQ ID NO: 25 - DNA, 97 bp, synthetic construct
  source 1..97: mol_type = other DNA
tcataaaaaatttatttgctttgtgagcggataacaattataatagattcaaatcggaggttctctaactagtatctctagagctaaggaggtaaat

SEQ ID NO: 26 - DNA, 36 bp, synthetic construct
  source 1..36: mol_type = other DNA
ttgacaattaatcatccggctcgtaatgtttgtgga

SEQ ID NO: 27 - protein, 369 aa, Bacillus subtilis
MKRHYEAVVIGGGIIGSAIAYYLAKENKNTALFESGTMGGRTTSAAAGMLGAHAECEERDAFFDFAMHSQRLYKGLGEELYALSGVDIRQHNGGMFKLAFSEEDVLQLRQMDDLDSVSWYSKEEVLEKEPYASGDIFGASFIQDDVHVEPYFVCKAYVKAAKMLGAEIFEHTPVLHVERDGEALFIKTPSGDVWANHVVVASGVWSGMFFKQLGLNNAFLPVKGECLSVWNDDIPLTKTLYHDHCYIVPRKSGRLVVGATMKPGDWSETPDLGGLESVMKKAKTMLPAIQNMKVDRFWAGLRPGTKDGKPYIGRHPEDSRILFAAGHFRNGILLAPATGALISDLIMNKEVNQDWLHAFRIDRKEAVQI

SEQ ID NO: 28 - DNA, 1110 bp, Bacillus subtilis
  source 1..1110: mol_type = other DNA
atgaaaaggcattatgaagcagtggtgattggaggcggaattatcggttccgcaattgcttattatttggcaaaggaaaacaaaaacaccgcattgtttgaaagcggaacaatgggcggcagaacgacaagtgccgctgccggaatgctgggcgcccatgccgaatgcgaggaacgtgacgcgttttttgatttcgctatgcacagtcagcgtctgtacaaaggtcttggagaagagctttatgcattatccggtgtggatatcaggcagcataacggcggtatgtttaagcttgcattttctgaagaagatgtgctgcagctgagacagatggacgatttggactctgtcagctggtattcaaaagaagaggtgttagaaaaagagccgtatgcgtctggtgacatctttggtgcatcttttattcaggatgatgtgcatgtggagccttattttgtttgcaaggcatatgtgaaagcagcaaaaatgcttggggcggagatttttgagcatacgcccgtcctgcatgtcgaacgtgacggtgaagccctgttcatcaagacccctagcggagacgtatgggctaatcatgttgtcgttgccagcggggtgtggagcggaatgttttttaaacagcttggactgaacaatgcttttctccctgtaaaaggggagtgcctgtccgtttggaatgatgatatcccgctgacaaaaacgctttaccatgatcactgctatatcgtaccgagaaaaagcggaagactggttgtcggcgcgacaatgaagccgggggactggagtgaaacaccggatcttggcggattggaatctgttatgaaaaaagcaaaaacgatgctgccggctatacagaatatgaaggtggatcgtttttgggcgggactccgtccgggaacaaaggatggaaaaccgtacatcggcagacatcctgaggacagccgtattttatttgcggctggccatttcagaaacgggatcctgcttgctcccgcaacgggcgctttgatcagtgatctcatcatgaataaagaggtcaaccaagactggctgcacgcattccgaattgatcgcaaggaggcggttcagatatga

SEQ ID NO: 29 - protein, 365 aa, Pseudomonas putida
MSKQVVVVGGGVIGLLTAFNLAAKVGQVVVCDQGEVGRESSWAGGGIVSPLYPWRYSPAVTALAHWSQDFYPQLGERLFASTGVDPEVHTTGLYWLDLDDEAEALAWAAREQRPLSAVDISAAYDAVPVLGPGFKHAIYMAGVANVRNPRLVKSLKAALLALPNVSLREHCQITGFVQEKGRVTGVQTADGVLAADEVVLSAGAWSGDLLRTLGLELPVEPVKGQMILFKCAEDFLPSMVLAKGRYAIPRRDGHILVGSTLEHAGYDKTPTADALESLKASAVELLPELEGATVVAHWAGLRPGSPEGIPYIGPVPGHEGLWLNCGHYRNGLVLAPASCQLFTDLLTGAEPIIDPAPYAPEGRLG

SEQ ID NO: 30 - DNA, 1098 bp, Pseudomonas putida
  source 1..1098: mol_type = other DNA
atgagcaagcaagtagtggtggttggcggcggggtgatcggcctgctgacggcattcaacctggcagccaaggtcggccaggtggtggtgtgcgatcagggcgaggttggccgagagtcgtcctgggctggcggaggcattgtctcgccgttgtacccgtggcgctacagccccgccgtgaccgctctggcgcactggtcgcaggacttttatccacagctgggcgagcgtctgttcgccagcaccggtgtcgacccggaagttcacaccaccgggctgtactggctcgacctggacgacgaggccgaagcgttggcctgggccgcgcgcgagcagcggccgctcagtgccgtggatatttcggcggcctacgatgcggtgccggtgctgggcccgggcttcaagcacgccatctacatggctggtgtggccaatgtgcgcaacccacgcttggtcaagtcgctgaaggcggcgttgctggcgctgccgaacgtgagcttgcgcgaacactgccagatcaccggcttcgtgcaggaaaaggggcgcgtgactggtgtacagacggctgatggcgtactggcggcggatgaggttgtgctcagcgcaggcgcctggagtggcgacctgttgcgtacccttggccttgaactgccggtggagcccgtgaagggccagatgattctgttcaagtgtgctgaagacttcttgccgagcatggtgctggccaaggggcgctatgcaatcccgcgacgtgacgggcacattctggtagggagcacgctggaacacgccgggtatgacaagaccccgaccgcggatgcgctggaaagcctgaaggcatcggcggtggagttgttgcctgagcttgaaggcgcgacagtggtggcgcactgggcgggactgcggccggggtcgccagaaggcatcccttacattgggcctgtgccagggcatgaagggttgtggctgaactgcgggcattatcgcaatgggctggtgctggcgccggcttcgtgccagctgtttaccgatttgctgacaggggccgagccgattatcgacccggcgccgtatgcgccagaagggcgtttgggctga

SEQ ID NO: 31 - protein, 162 aa, Escherichia coli
MRLTSKGRYAVTAMLDVALNSEAGPVPLADISERQGISLSYLEQLFSRLRKNGLVSSVRGPGGGYLLGKDASSIAVGEVISAVDESVDATRCQGKGGCQGGDKCLTHALWRDLSDRLTGFLNNITLGELVNNQEVLDVSGRQHTHDAPRTRTQDAIDVKLRA

SEQ ID NO: 32 - DNA, 489 bp, Escherichia coli
  source 1..489: mol_type = other DNA
atgagactgacatctaaagggcgctatgccgtgaccgcaatgcttgacgttgcgctcaactctgaagcgggcccggtaccgttggctgatatttccgaacgtcagggaatttccctttcttatctggaacaactgttttcccgtctgcgtaaaaatggtctggtttccagcgtacgtggaccaggcggtggttatctgttaggcaaagatgccagcagcatcgccgttggcgaagtaattagcgccgttgacgaatctgtagatgccacccgttgtcagggtaaaggcggctgccagggcggcgataaatgcctgacccacgcgctgtggcgtgatttgagcgaccgtctcaccggttttctcaacaacattactttaggcgaactggttaataaccaggaagtgctggatgtgtctggtcgtcagcatactcacgacgcgccacgcacccgcacacaagacgcgatcgacgttaagttacgcgcttaa

SEQ ID NO: 33 - protein, 99 aa, Escherichia coli
MREIAQQTEQEVVEVETVNGFGPNDVILDIRSIDEQEDKPLKVEGIDVVSLPFYKLSTKFGDLDQNKTWLLWCERGVMSRLQALYLREQGFNNVKVYRP

SEQ ID NO: 34 - DNA, 300 bp, Escherichia coli
  source 1..300: mol_type = other DNA
atgcgcgaaatcgcccagcagaccgagcaggaagtggtggaagtggaaaccgtcaatggcttcggcccgaacgacgtgatcctcgatatccgttctatcgatgaacaggaagataagccactgaaagtcgaagggattgatgtggtttctctgccgttctataaactgagcaccaaatttggcgatctcgaccagaacaaaacctggctgctgtggtgtgagcgcggggtgatgagccgtctgcaggcgctctatctgcgcgagcagggctttaacaatgtgaaggtatatcgcccgtaa

SEQ ID NO: 35 - protein, 484 aa, Pseudomonas putida
MKLIVKVFPEITIKSRPVRKRFIRQLGKNIRNVLKDLDPELAVDGVWDNLEVVTRVEDEKVQREMIERLTCTPGITHFLQVEEYPLGDFDDIVAKCKHHFGHLLAGKHFAVRCKRGGHHDFTSMDVDRYVGSQLRQQCGAAGIELKKPEVLVRIEIRDQRLYVIHNQHNGIGGYPLGALEQTLVLMSGGFDSTVAAYQMMRRGLMTHFCFFNLGGRAHELGVMEVAHYLWKKYGSSQRVLFISVPFEEVVGEILNKVDNSYMGVTLKRMMLRGAAHMADRLQIDALVTGEAISQVSSQTLPNLSIIDSATDKLVLRPLLASHKQDIIDQATEIGTADFAKHMPEYCGVISVNPTTHAKRHRMEHEEKQFDMAVLERALERAKFISIDHVIDELGKDIEIEEVAEALPGQIVIDIRHPDAQEDEPLVLEGIEVQAMPFYAINSKFKHLDPTRQYLLYCDKGVMSRLHAHHLLSEGHANVRVYRPT

SEQ ID NO: 36 - DNA, 1455 bp, Pseudomonas putida
  source 1..1455: mol_type = other DNA
atgaaacttatcgtcaaagtcttcccagaaatcaccatcaaaagccggccggtgcgcaagcgcttcatccgccagcttggcaagaacatccgcaacgtgctcaaggatctcgaccctgagctcgcggtcgatggtgtctgggacaatctcgaggtggtcacccgcgtcgaagacgaaaaagtccagcgcgagatgatcgaacgcctcacctgcaccccgggtatcacccacttcctgcaggtagaggaatacccgctgggtgacttcgacgacatcgtcgccaagtgcaagcaccacttcggccacctgctggccggcaagcacttcgccgtgcgctgcaagcgcggtggccaccatgacttcacctcgatggacgtcgaccgttacgtcggcagccaactgcgtcagcagtgtggcgccgccgggatcgagctgaaaaagcctgaagtgctggtgcgcatcgaaatccgcgaccagcgcctgtacgtgatccacaaccagcacaatggcatcggcggttatccgctgggtgccctggagcagactctggtgctgatgtccggtggtttcgactccaccgttgcggcctaccagatgatgcgccgcggcctgatgacccacttctgcttcttcaacctcggcggccgtgcccacgagctgggcgtaatggaagtggcccattacctgtggaaaaaatacggcagcagccagcgcgtactgttcatcagcgtgccgttcgaagaagtggttggcgagatcctcaacaaggtcgacaacagctacatgggcgtgaccctcaagcgcatgatgctgcgcggcgccgcccatatggccgaccgcctgcagattgacgcgctggtgaccggcgaagcgatttcccaggtgtccagccagaccctgccgaacctgtcgatcatcgactcggccaccgacaagctggtgctgcgcccgctgctggccagccacaagcaggacatcatcgaccaggccaccgaaatcggtaccgcggactttgccaagcacatgccggaatactgcggcgtgatctcggtaaacccgaccacccatgccaagcgtcaccgcatggagcacgaagaaaagcagttcgacatggccgtgctggagcgcgctcttgagcgcgccaagttcatttccatcgatcatgtgatcgatgagctgggcaaggacatcgaaatcgaggaagtggccgaggcgctgccaggccagatcgtcatcgacattcgccaccccgatgcccaggaagacgaacctctggtgctggaaggtatcgaagtccaggccatgccgttctacgccatcaacagcaagttcaagcacctggaccccacgcgccagtacttgctgtattgcgacaagggtgtgatgagccgtttgcacgcacaccatctgctcagtgagggacatgccaatgtgcgtgtttatcgtccgacataa

SEQ ID NO: 37 - protein, 484 aa, Azotobacter vinelandii
MKLIVKVFPEITIKSPPVRKRFIRQLAKNIRTVLRDLDPELAVTGVWDNLEVETAVEKPRLLREMIERLCCTPGIAHFLEVHEYPLGDLDDILEKCKRHYAGQLPGKIFAVRCKRAGRHPFTSMEVERYVGGCLRQQCGAAGVSLSAPEVEVRMEIRDQRLFVVHRQHDSIGGYPLGALEQTLVLMSGGFDSTVAAFQTMRRGLMTHFCFFNLGGRAHELGVMEVAHYLWQKYGSSQRVLFVSVPFEEVVGEILGKVDNSQMGVILKRMMLRAATRVAERLKIDALVTGEAISQVSSQTLPNLSVIDSATDMLVLRPLIASHKQDIIDTATRIGTAEFARHMPEYCGVISVNPTTRARRDRIDYEERQFDMAILERALERAHLVPIDRVIDELGEEICVEEVREALAGQIVLDIRHPEAVEDGPLELPGIEVRALPFYALNNRFKELDSNRQYLLYCDKGVMSRLHAHHLLSEGHANVRVYRPA

SEQ ID NO: 38 - DNA, 1455 bp, Azotobacter vinelandii
  source 1..1455: mol_type = other DNA
atgaaactcatcgtcaaggtcttcccggaaatcaccatcaagagcccgccggtgcgcaagcgcttcattcgccaactggcgaagaacatccgcaccgtgctgcgcgacctggatccggagctggcggtgacgggcgtgtgggacaacctcgaggtggagaccgcggtcgagaagccgcgcctgttgcgcgagatgatcgagcggctgtgctgcacgcccggtatcgcccacttcctcgaggtgcacgagtatcccctgggcgacctcgacgacatcctggagaagtgcaagcgccactacgccgggcaactgcccggcaagatcttcgccgtgcgctgcaagcgcgccggcaggcatcccttcacctcgatggaggtggagcgctacgtcggcggctgcctgcgccagcagtgcggtgccgcgggcgtctcgctgagcgctccggaagtggaagtgcgcatggagatccgcgaccagcgcctgttcgtcgtccaccgtcagcacgacagcatcggcggttacccgctgggcgccctggagcagaccctggtgctgatgtccggcggcttcgactccaccgtggcggctttccagacgatgcgccgcgggctgatgacccatttctgcttcttcaacctgggcgggcgcgcccacgagctgggggtgatggaagtcgcccactacctctggcagaagtacggcagctcgcagcgcgtgctgttcgtcagcgtgcccttcgaggaggtggtcggcgagatcctcggcaaggtcgacaacagccagatgggcgtgatcctgaagcgcatgatgctgcgcgccgccacgcgcgtcgccgagcgcctgaagatcgacgcgctggtcaccggcgaagccatctcccaggtgtccagccagaccctgccgaacctctcggtgatcgattcggccaccgacatgctggtgctgcgcccactgatcgccagccacaagcaggacatcatcgacaccgcgacgcgcatcggcaccgccgagttcgcccggcacatgcccgaatactgcggggtgatctcggtgaatccgaccacccgcgccaggcgtgaccgcatcgattacgaggagcgccagttcgacatggcgatcctcgagcgcgcgctggagcgcgcccacctggtgccgatcgaccgggtgatcgacgaactgggcgaggagatttgcgtggaggaggtccgcgaggctctggccggacagatcgtcctcgacatccgccatccggaggcggtcgaggacgggccgctggagttgcccggcatcgaggtgcgggcgctgcctttctatgcgctgaacaaccgcttcaaggaactggacagcaaccgccaataccttctttattgcgacaagggtgtcatgagccgcctgcatgctcatcacctgctgagcgagggacatgccaatgtgcgcgtttatcgtccggcatag

SEQ ID NO: 39 - protein, 484 aa, Shewanella baltica
MKFIVKLYPEIMMKSKPVRMRFTKMLETNIRNVLKKVDEDAKVQRQWDRIMVMVPKDKPELAQAFGERLACIPGIAHVVQVDEYSFESVDDIYQQVLPVYRDQLAGKTFCVRVKRTGDHDFNSIEVERYVGGGLNQFTDALGVRLKNPDITVNLEIERDNLYMVTKRIEGLGGFPMATQEDVLSLISGGFDSGVSSYQFIKKGARTHYCFFNLGGAQHEIGVKQVAYHLWKTYGESHKVKFISVPFEPVVAEILERIDNGQMGVVLKRMMMRTAARIADRMGIQALVTGESLGQVSSQTLTNLNVIDRCTELLILRPLIAMDKQDIINESRKIGTEDFAKSMPEYCGVISQKPTVKAVLAKVEAEEKKFSEDLIDQIIAQSVTIDIREIAEQMDTRITETETVASIDTNQVVIDIRAPEEEESKPLQIEGIEIKRIPFFKLATQFADLDKQKTYLLYCERGVMSKLQALYLIEQGYTNVKVYRP

SEQ ID NO: 40 - DNA, 1455 bp, Shewanella baltica
  source 1..1455: mol_type = other DNA
atgaagtttattgtaaagctgtacccagaaatcatgatgaagagcaaacccgtaaggatgcgcttcaccaaaatgctcgaaaccaacatacgtaatgtgctcaaaaaagtcgacgaagacgctaaagttcaacgtcaatgggaccgcattatggtaatggtgccaaaggataagcctgaattggcacaagccttcggtgagcgtctggcgtgtattcctggcattgcacacgtagtgcaagtggatgagtacagctttgaatctgtggacgatatttatcagcaagtcttgcctgtttaccgtgaccaactcgcgggtaaaaccttctgcgtgcgagtgaagcgcactggcgatcacgattttaattctatcgaagtggagcgttatgttggcggtggcttaaatcagtttaccgatgctttaggtgtgcgtttaaagaatcccgatattaccgttaaccttgaaattgaacgcgataatctttacatggtcactaagcgtattgaaggcttaggtggtttcccgatggcgactcaggaagatgtgttatctttgatttcaggtggttttgactctggtgtgtctagctatcaatttatcaaaaagggcgcgcgtacccattactgcttctttaatctgggcggcgcgcagcatgaaattggcgtaaaacaagtggcttaccatttgtggaaaacctacggcgaatcccacaaggtgaagtttatctctgtgccttttgagcctgtggtcgctgagattttagagcgtatcgataatggccaaatgggcgttgtgctcaagcgtatgatgatgcgcactgcggcgcgtattgccgaccgtatgggcattcaagccttggtgaccggtgaaagtttaggccaagtctctagccaaacgctgaccaacttaaacgtgattgaccgctgtactgagctgctgattttacgtccgctgatcgccatggataaacaagacattatcaacgagagccgtaaaatcggcaccgaagattttgccaagtctatgcctgaatattgtggcgtgatttcgcaaaagccaacggttaaagcggtattagcgaaagttgaagcggaagagaagaagttctctgaagatttgatcgatcaaatcatcgcccagtctgtcactattgatatcagggagatcgcagaacaaatggatactcggattactgagactgaaacggtcgccagtattgataccaaccaagtggtcattgatattcgcgcccctgaagaagaggaaagcaaaccgttgcaaattgaaggcattgagattaaacgtatcccattcttcaagttagcgactcaatttgccgatctcgataagcaaaaaacctatttgctgtattgtgagcgtggggtgatgagtaaattgcaggcattgtatttaatcgagcaaggctacactaacgtcaaagtgtatcgcccataa

SEQ ID NO: 41 - protein, 401 aa, Bacillus subtilis
MNYDHILIRFGEISTKGKNRKSFIERLKQNIRLVLKDYPNLKYFSNRDRMTITLNGEDPEALFPHLKQVFGIQSFSLAIKCDSRLDDIKATALKAIKDQYKPGDTFKVATKRAYKQFELDTNQMNAEIGGHILRNTEGLTVDVRNPDIPLRIEIREEATFLTIRDEKGAGGLPVGSAGKAMLMLSGGFDSPVAGFYAMKRGLSVEAVHFFSPPYTSERAKQKVMDLAKCLSRFGGSMTLHIVPFTKTQELIQKQIPENYTMTATRRLMLQIADRIREKRNGLAIITGESLGQVASQTLESMYAINAVTSTPILRPLIAMDKTEIIEKSREIGTYETSIQPFEDCCTIFTPPSPKTRPKKEKIEHFESFVDFEPYIQEAVDNIETMTLYSEQEANDKFAELF

SEQ ID NO: 42 - DNA, 1206 bp, Bacillus subtilis
  source 1..1206: mol_type = other DNA
atgaattacgatcatatattaattcgttttggggaaatttcgaccaaaggcaaaaacagaaaaagctttattgagcgcttaaaacaaaacatcagactcgttttgaaagactatccgaatttaaaatacttttccaacagggatcgtatgacgattacattaaatggagaggatcctgaggctctttttcctcatttaaagcaagtgtttgggattcaaagcttcagcctggcaattaagtgtgacagccgccttgatgatattaaggcaaccgcactaaaggctatcaaagatcagtataaaccaggtgatacgtttaaagtcgcgacaaagagagcgtataagcaatttgaattagatacaaaccagatgaacgctgaaatcggcggccatattttgcgaaatacagaaggattgacggtcgatgttcgcaacccagacatcccgctcagaatcgaaatcagggaagaagcaacatttttgacaattcgtgatgaaaaaggtgcaggcgggctgccagttggttcagcaggaaaagcgatgctgatgctttctggcggttttgacagccctgtggccggtttttatgcgatgaaacggggattgtcggtagaagctgttcatttcttcagcccgccatacacaagcgagcgcgcaaagcaaaaggtaatggatctggcgaaatgtctgtcccgttttgggggaagcatgacacttcatattgttccatttacaaaaacgcaagagctcatccaaaaacaaattcctgaaaattacacaatgacggcaacccgccgcctcatgcttcagatagccgacagaatcagagaaaagagaaacgggcttgccattattacgggagaaagcctcggtcaggtagcgagccaaacgcttgaaagcatgtatgcaattaatgcggtgacgtcaacaccgattttgcgtccgctaatcgccatggacaaaacagaaatcatcgaaaaatcacgggaaattggcacatatgagacaagtattcagccgtttgaagactgctgcacgatctttacaccgccaagcccgaaaacacgccctaaaaaagagaaaattgaacactttgaaagctttgttgatttcgagccgtatattcaagaagcggtagataacattgagacaatgaccctttatagcgaacaagaagctaacgataaattcgctgaactcttttaa

SEQ ID NO: 43 - DNA, 37 bp, synthetic construct
  source 1..37: mol_type = other DNA
ttgacatcgcatctttttgtacctataatagattcat

SEQ ID NO: 44 - DNA, 37 bp, synthetic construct
  source 1..37: mol_type = other DNA
ttgacatcgcatctttttgtacctataatgtgtggat

SEQ ID NO: 45 - DNA, 45 bp, synthetic construct
  source 1..45: mol_type = other DNA
tcataaaaaatttatttgctttgtgagcggataacaattataata

SEQ ID NO: 46 - DNA, 52 bp, synthetic construct
  source 1..52: mol_type = other DNA
gattcaaatcggaggttctctaactagtatctctagagctaaggaggtaaat

SEQ ID NO: 47 - DNA, 46 bp, synthetic construct
  source 1..46: mol_type = other DNA
aaaagagtattgacttcgcatctttttgtacctataatagattcat

SEQ ID NO: 48 - DNA, 30 bp, Synthetic construct
  source 1..30: mol_type = other DNA
attaatcatccggctcgtagtgtttgtgga